(19) United States
(12) Patent Application Publication
(10) Pub. No.: US 2021/0228643 A1
(43) Pub. Date: Jul. 29, 2021

(54) N VIVO DELIVERY SYSTEM OF THE GENOME DNA MODIFYING ENZYMES AND THE USE THEREOF

(71) Applicants: UNIWERSYTET JAGIELLONSKI, Krakow (PL); ALBERT-LUDWIGS UNIVERSITY FREIBURG, Freiburg (DE)

(72) Inventors: Sylwia BOBIS-WOZOWICZ, Brzezie (PL); Ewa ZUBA-SURMA, Krakow (PL); Toni CATHOMEN, Freiburg (DE)

(21) Appl. No.: 17/262,789

(22) PCT Filed: Jul. 26, 2018
(86) PCT No.: PCT/IB2018/055591

§ 371 Date: Jan. 25, 2021

(51) Int. Cl. A61K 35/28; A61K 38/46

(52) U.S. Cl. CPC A61K 35/28; A61K 38/465

(57) ABSTRACT

The present invention relates to the in vivo delivery system of the DNA modifying enzymes comprising the stem cells-derived extracellular vesicles collected in serum-free conditions, characterized in that the said extracellular vesicles are the population of extracellular vesicles defined by the expression of surface markers. Moreover, the present invention is dedicated for use in a treatment of a genetic disease and/or disorder.

### FIELD OF THE INVENTION

The present invention relates to the field of molecular biology and medicine. It concerns the in vivo delivery system of the DNA modifying enzymes. More precisely, the present invention enables delivery of the DNA modifying enzymes for the purpose of precise genome editing in vivo, the said delivery using stem cells-derived extracellular vesicles (EVs) collected in serum-free conditions and characterized by the expression of surface markers. Preferably the population of extracellular vesicles is defined by the expression of surface markers: CD90, CD105, CD147, CD309 and lack of expression CD45. Furthermore, the present invention concerns the use of said delivery system in a treatment of genetic diseases and/or disorders.

### BACKGROUND OF THE INVENTION

Hybrid nucleases, including ZFN, TALEN and the CRISPR/Cas9 system, constitute indispensable tools in genome editing, due to precise activity at strictly defined and pre-selected genomic loci (see, for example, Yin H, Kauffman K J, Anderson D G. Delivery technologies for genome editing. Nat Rev Drug Discov. 2017, 16(6):387-399 and Nelson C E, Gersbach C A. Engineering Delivery Vehicles for Genome Editing. Annu Rev Chem Biomol Eng. 2016 Jun. 7; 7:637-62 and references included therein). The use of designer nucleases outperforms other technologies for genome engineering, such as viral vectors, which carry considerable risk of genetic alterations of target cells and, in a consequence, may lead to malignant transformation if inserted into inappropriate sites of the genome (see, Kustikova O, Fehse B, Modlich U, Yang M, Düllmann J, Kamino K, von Neuhoff N, Schlegelberger B, Li Z, Baum C. Clonal dominance of hematopoietic stem cells triggered by retroviral gene marking. Science. 2005 May 20; 308(5725):1171-4 and Baum C, Modlich U, Göhring G, Schlegelberger B. Concise review: managing genotoxicity in the therapeutic modification of stem cells. Stem Cells. 2011 October; 29(10):1479-84). Thus, development of precise genome editing technologies enabled improving safety profile of gene therapy clinical trials and holds great promise for regenerative medicine in the future (as described in Cornu T I, Mussolino C, Cathomen T. Refining strategies to translate genome editing to the clinic. Nat Med. 2017 Apr. 3; 23(4):415-423 and Abou-El-Enein M, Cathomen T, Ivies Z, June C H, Renner M, Schneider C K, Bauer G. Human Genome Editing in the Clinic: New Challenges in Regulatory Benefit-Risk Assessment. Cell Stem Cell. 2017 Oct. 5; 21(4):427-430).

In most instances, designer nuclease activity is needed only transiently—just long enough to cleave the target site. Prolonged expression increases the risk of so-called off-target activity, which can be genotoxic. Transient expression can be achieved ex vivo by delivering the nucleases in the forms of mRNA or protein (for review, see, Bobis-Wozowicz S, Galla M, Alzubi J, Kuehle J, Baum C, Schambach A, Cathomen T. Non-integrating gamma-retroviral vectors as a versatile tool for transient zinc-finger nuclease delivery. Sci Rep. 2014 Apr. 11; 4:4656; Cai Y, Bak R O, Mikkelsen J G. Targeted genome editing by lentiviral protein transduction of zinc-finger and TAL-effector nucleases. Elife. 2014 Apr. 24; 3:e01911; Liu J, Gaj T, Yang Y, Wang N, Shui S, Kim S, Kanchiswamy C N, Kim J S, Barbas C F 3rd. Efficient delivery of nuclease proteins for genome editing in human stem cells and primary cells. Nat Protoc. 2015 November; 10(11):1842-59; Jia J, Bai F, Jin Y, Santostefano K E, Ha U H, Wu D, Wu W, Terada N, Jin S. Efficient Gene Editing in Pluripotent Stem Cells by Bacterial Injection of Transcription Activator-Like Effector Nuclease Proteins. Stem Cells Transl Med. 2015 August; 4(8):913-26 and Hashimoto M, Takemoto T. Electroporation enables the efficient mRNA delivery into the mouse zygotes and facilitates CRISPR/Cas9-based genome editing. Sci Rep. 2015 Jun. 11; 5:11315). For in vivo delivery, however, these platforms are not efficient. Moreover, gene delivery via liposomes or other artificial nano-vesicles seem to be inefficient and may trigger an immune response in the host (see, for example, Saffari M, Moghimi H R, Dass C R. Barriers to Liposomal Gene Delivery: from Application Site to the Target. Iran J Pharm Res. 2016 Winter; 15(Suppl):3-1712,13 and Zylberberg C, Gaskill K, Pasley S, Matosevic S. Engineering liposomal nanoparticles for targeted gene therapy. Gene Ther. 2017 August; 24(8):441-452), which decreases expected biological effect. Therefore, the use of EVs, which play an important role in cell-to-cell communication, constitute an attractive technological solution to deliver designer nucleases.

EVs are natural nanovesicles released by any type of cells in activated or steady-state conditions. They are composed of a cellular membrane enclosing cytosolic components. By transferring their bioactive cargo, including small RNAs, messenger RNAs, proteins and lipids, EVs may influence fate decisions of the acceptor cells (for review, see, Camussi G, Deregibus M C, Bruno S, Cantaluppi V, Biancone L. Exosomes/microvesicles as a mechanism of cell-to-cell communication. Kidney Int. 2010 November; 78(9):838-48 and Quesenberry P J, Goldberg L R, Aliotta J M, Dooner M S, Pereira M G, Wen S, Camussi G (2014) Cellular phenotype and extracellular vesicles: basic and clinical considerations. Stem Cells Dev 23:1429-1436 and references included therein).

Based on their size and origin, EVs can be divided into: i) exosomes (approx. 30-100 nm in diameter), which are derived from the endosomal compartment and express on their surface typical exosomal markers, such as CD9, CD63, CD81) and ii) microvesicles (ectosomes or shedding vesicles, approx. 100-1000 in diameter), which are generated by budding of the cellular membrane and are characterized by the presence of surface markers typical for their parental cells (see, for example, Quesenberry P J, Goldberg L R, Aliotta J M, Dooner M S, Pereira M G, Wen S, Camussi G (2014) Cellular phenotype and extracellular vesicles: basic and clinical considerations. Stem Cells Dev 23:1429-1436).

Utility of EVs derived from various cell populations, including stem cells, in transferring biological cargo to other cells has gain considerable interest in recent years. There have been attempts to use EVs for promoting neurogenesis (US 2017/0368103 A1); treatment of neuronal diseases (KR 20130116552 A); in wound healing (WO 2015/052527 A1); treating cancer (WO 2015/052526), pain treatment (US 2018/0036348 A1) and many others (for example, Keshtkar S, Azarpira N, Ghahremani M H. Mesenchymal stem cell-derived extracellular vesicles: novel frontiers in regenerative medicine. Stem Cell Res Ther. 2018 Mar. 9; 9(1):63 and BjØrge I M, Kim S Y, Mano J F, Kalionis B, Chrzanowski W. Extracellular vesicles, exosomes and shedding vesicles in regenerative medicine—a new paradigm for tissue repair. Biomater Sci. 2017 Dec. 19; 6(1):60-78). WO 2016187717 A1 disclosing a particular EV fraction of exosomes has also been proposed as a tool for designer nucleases delivery to edit genetic defects in the treatment of genetic diseases. However, antigen-defined stem cell-derived EVs (in particular ectosomes) has never been considered.

In this work, DN-containing EVs were harvested from stem cells, which constitute clinically relevant biological material and are applied in numerous regenerative therapies worldwide. However, EVs due to their acellular nature and lack of nucleus, are considered to be safer than their cellular counterparts, because their use is not associated with tumorigenesis, which has already been confirmed (see, Adamiak M, Cheng G, Bobis-Wozowicz S, Zhao L, Kedracka-Krok S, Samanta A, Karnas E, Xuan Y T, Skupien-Rabian B, Chen X, Jankowska U, Girgis M, Sekula M, Davani A, Lasota S, Vincent R J, Sarna M, Newell K L, Wang O L, Dudley N, Madej a Z, Dawn B, Zuba-Surma E K. Induced Pluripotent Stem Cell (iPSC)-Derived Extracellular Vesicles Are Safer and More Effective for Cardiac Repair Than iPSCs. Circ Res. 2018 Jan. 19; 122(2):296-309).

The whole fraction of EVs was used in this study, with the emphasis on ectosomes (shedding vesicles), characterized by the presence of surface markers typical for stem cells, including CD90, CD105, CD309, CD147 and lack of expression of a hematopoietic antigen CD45. Importantly, EVs were collected in a serum-free medium, which is of great significance for future clinical applications to treat human diseases. In our previous work we have shown that media composition for stem cell culture impacts on biological properties of their EVs (see, Bobis-Wozowicz S, Kmiotek K, Kania K, Karnas E, Labedz-Maslowska A, Sekula M, Kedracka-Krok S, Kolcz J, Boruczkowski D, Madeja Z, Zuba-Surma E K. Diverse impact of xeno-free conditions on biological and regenerative properties of hUC-MSCs and their extracellular vesicles. J Mol Med (Berl). 2017 February; 95(2):205-220).

By employing a model system based on green fluorescence, we have shown activity of designer nucleases delivered to target cells by our new technology both in vitro, on a cell line and primary cells, and in vivo, in experimental animals. Importantly, efficacy of our approach reached almost 50% of genome edited cells in certain tissues in a mouse model, particularly in the liver.

### SUMMARY OF THE INVENTION

The invention provides a novel method of in vivo genome editing with the use of hybrid nucleases, such as ZFN, TALEN and the CRISPR/Cas9 system, delivered to target cells by EVs derived from stem cells in serum-free conditions and defined as the RNA-containing vesicles expressing stem cells surface markers: CD90, CD105, CD147, CD309 (in more than 20% of RNA-positive events) and lack of expression CD45m is presented here.

Specifically, invention provides an in vivo delivery system of the DNA modifying enzymes comprising the stem cells-derived extracellular vesicles collected in serum-free conditions, characterized in that the said extracellular vesicles are the population of extracellular vesicles defined by the expression of surface markers.

Preferably, extracellular vesicles of the delivery system are microvesicles.

Preferably, the population of extracellular vesicles is defined by the expression of surface markers: CD90, CD105, CD147, CD309 and lack of expression CD45.

Preferably, at least 10% of the extracellular vesicle's population contains mRNA. More preferably, mRNA is selected from the mRNA of the hybrid nucleases, recombinases, integrase, resolvases/invertases and transposases.

Preferably, the hybrid nucleases are selected from the Zinc Finger Nucleases (ZFN), Transcription Activator-Like Effector Nucleases (TALEN) and the CRISPR/Cas9 and the meganucleases. More preferably, the recombinases are selected from the Flp, Cre, Dre, KD, B2, B3, RadA, RAD51, RecA and Dmc1; the integrases are selected from the λ, HK022, HP1, ϕC31, Bxb1 and R4; and the resolvases/invertases are selected from the γδ, ParA, Tn3 and Gin. Preferably, at least 20% of the RNA-positive population presents the surface antigens: CD90, CD105, CD147, CD309 and has no expression of CD45.

Preferably, the stem cell is selected from the mesenchymal stem cell and pluripotent stem cell. More preferably, the mesenchymal stem cell is a umbilical cord-derived mesenchymal stem cell, and the pluripotent stem cell is the induced pluripotent stem cell.

The invention provides the delivery system defined herein for use in a treatment of a genetic disease and/or disorder.

Preferably, the genetic disease and/or disorder for which the delivery system according to the invention will be beneficial, is a mammalian genetic disease and/or disorder. More preferably, the genetic disease and/or disorder for which the delivery system according to the invention will be beneficial, is selected from the liver-associated diseases, spleen-associated diseases, lung-associated diseases, heart-associated diseases and kidney-associated diseases.

Specifically, the liver-associated disease, for which the delivery system of the invention will be useful, is selected from the following list and/or relates to disfunction in the following genes: fatty liver disease, nonalcoholic, NAFLD, polycystic kidney disease with or without polycystic liver disease; PKD, glycogen storage disease, GSD, thyroid dyshormonogenesis 4; TDH4, hepatocellular carcinoma, hepatoblastoma, Niemann-Pick disease, NPD, alpha-1-antitrypsin deficiency; A1ATD, mitochondrial DNA depletion syndrome 4A (ALPERS type); MTDPS4A, maple syrup urine disease; MSUD, pyruvate kinase, liver and red blood cell; PKLR, alkaline phosphatase, liver; ALPL, Wilson disease, visceral steatosis, congenital, serpin peptidase inhibitor, clade A, member 1; SERPINA1, Huntington disease; HD, hemochromatosis, type 1; HFE1, aldehyde dehydrogenase 2 family; ALDH2, Fabry disease, major histocompatibility complex, class I, B; HLA-B, preeclampsia/eclampsia 1; PEE1, hypertension, cystic fibrosis; CF, telangiectasia, hereditary hemorrhagic, type 1; HHT1, infantile liver failure syndrome 1; ILFS1, tyrosinemia, type I; TYRSN1, Tay-Sachs disease; TSD, gap junction protein, beta-1; GJB1, apolipoprotein E; APOE, glucokinase; GCK, Menkes disease, Pendred syndrome; PDS, polycystin 1; PKD1, thyroid dyshormonogenesis 3; TDH3, HNF1 homeobox B; HNF1B, phosphorylase kinase, testis/liver, gamma-2; PHKG2, interstitial lung and liver disease; ILLD, thyroid dyshormonogenesis 2A; TDH2A, phosphorylase kinase, liver, alpha-2 subunit; PHKA2, thyroid dyshormonogenesis 1; TDH1, glucosidase, beta, acid; GBA, glutathione S-Transferase, MU-1; GSTM1, phenylketonuria; PKU, hyperphenylalaninemia, mitochondrial DNA depletion syndrome 6 (hepatocerebral type); MTDPS6, infantile sialic acid storage disease; ISSD, Sandhoff disease, C-type lectin domain family 4, member M; CLEC4M, cadherin 1; CDH1, solute carrier family 2 (facilitated glucose transporter), member 2; SLC2A2, Chanarin-Dorfman syndrome; CDS, hepatic adenomas, familial, cholestasis, progressive familial intrahepatic, 1; PFIC1, arginase 1; ARG1, peroxisome biogenesis disorder 1B; PBD1B, adrenoleukodystrophy; ALD, infantile liver failure syndrome 2; ILFS2, protein kinase C substrate, 80-KD, heavy chain; PRKCSH, Gaucher disease, GD, hepcidin antimicrobial peptide; HAMP, myoclonic epilepsy of LAFORA epilepsy, lysosomal acid lipase deficiency, Caroli disease, glycogen synthase 2; GYS2, amyloid beta A4 precursor protein; APP, hemophilia B; HEMB, liver fibrocystic disease and polydactyly, nephropathy, progressive tubulointerstitial, with cholestatic liver disease, Gallbladder disease 1; GBD1, lymphoproliferative syndrome, X-linked, 1; XLP1, phosphofructokinase, liver type; PFKL, cystic fibrosis transmembrane conductance regulator; CFTR, carnitine palmitoyltransferase I, liver; CPT1A, PKHD1 gene; PKHD1, O-phosphoserine tRNA-selenocysteine tRNA synthase; SEPSECS, cytochrome P450, family 4, subfamily F, polypeptide 2; CYP4F2, celiac disease, susceptibility to, 1; CELIAC1, Refsum disease, classic, Fanconi-Bickel syndrome; FBS, telangiectasia, hereditary hemorrhagic, type 2; HHT2, ATPase, Cu(2+)-transporting, beta polypeptide; ATP7B, interleukin 10; IL10, transforming growth factor, beta-1; TGFB1, tumor protein p53; TP53, peroxisome biogenesis disorder 1A (Zellweger); PBD1A, Alzheimer disease; AD, sphingomyelin phosphodiesterase 1, acid lysosomal; SMPD1, diabetes mellitus; DM, von Willebrand disease; VWD, chylomicron retention disease; CMRD, glucosidase, alpha, neutral AB; GANAB, Farber lipogranulomatosis; FRBRL, sickle cell anemia, hypercholesterolemia, transthyretin; TTR, Letterer-Siwe disease, hyperlipoproteinemia, graft-versus-host disease, polycystin 2; PKD2, mitochondrial complex I deficiency, inflammatory bowel disease (Crohn disease) 1; IBD1, parkin; PARK2, ATP-binding cassette, subfamily A, member 1; ABCA1, Alagille syndrome 1; ALGS1, von Hippel-Lindau syndrome; VHL, hemoglobin-beta locus; HBB, Creutzfeldt-Jakob disease; CJD, Salla disease; SD, synuclein, alpha; SNCA, cirrhosis, osteopetrosis, autosomal recessive 1; OPTB1, mucolipidosis II alpha/beta, UDP-glycosyltransferase 1 family, polypeptide A1; UGT1A1, renal cysts and diabetes syndrome; RCAD, phosphatase and tensin homolog; PTEN, immunodeficiency with hyper-IgM, type 1; HIGM1, polyglucosan body neuropathy, adult form; APBN, porphyria, congenital erythropoietic, superoxide dismutasE 1; SOD1, glycogen phosphorylase, liver; PYGL, facioscapulohumeral muscular dystrophy 1; FSHD1, insulin receptor substrate 1; IRS1, Hartnup disorder; HND, paraoxonase 1; PON1, nitric oxide synthase 3; NOS3, tumor necrosis factor; TNF, carbamoyl phosphate synthetase I; CPS1, solute carrier family 17 (acidic sugar transporter), member 5; SLC17A5, familial adenomatous polyposis 1; FAP1, Hermansky-Pudlak syndromE 1; HPS1, abetalipoproteinemia; ABL, serpin peptidase inhibitor, clade A, member 3; SERPINA3, Danon disease, Coach syndrome, glycogen phosphorylase, muscle; PYGM, NPC1 gene; NPC1, nuclear receptor subfamily 1, group H, member 3; NR1H3, hemophilia A; HEMA, peroxisome proliferator-activated receptor-gamma, coactivator 1, alpha; PPARGC1A, Tangier disease; TGD, prion protein; PRNP, peroxisome proliferator-activated receptor-gamma; PPARG, galactosidase, alpha; GLA, interleukin 6; IL6, leprosy, susceptibility to, 1; LPRS1, Friedreich ataxia 1; FRDA, glutamate pyruvate transaminase; GPT, survival of motor neuron 1; SMN1, cytochrome c oxidase, subunit 6A1; COX6A1, Charcot-Marie-Tooth disease; CMT, Parkinson disease, glutathione S-transferase, alpha-2; GSTA2, fructose-1,6-bisphosphatase 1; FBP1, adrenal hypoplasia, congenital; AHC, transmembrane protein 67; TMEM67, Darier-White disease; DAR, lecithin:cholesterol acyltransferase deficiency, growth factor, ERV1-like; GFER, gliomedin; GLDN, methionine adenosyltransferase I, alpha; MAT1A, transmembrane protein 59; TMEM59, Cowden syndrome 1; CWS1, 6-phosphofructo-2-kinase/fructose-2,6-bisphosphatase 1; PFKFB1, pleckstrin homology-like domain, family A, member 2; PHLDA2 (based on OMIM database https://www.omim.org/search/?index=entry&start=1&limit=10&sort=score+desc %2C+prefix_sort+desc&search=liver+genetic+disease).

Specifically, the spleen-associated disease, for which the delivery system of the invention will be useful, is selected from the following list and/or relates to disfunction in the following genes: chemokine, cxc motif, receptor 4; CXCR4, Niemann-Pick disease, type C1; NPC1, Niemann-Pick disease, type D, type B, type F, type A, polycystic kidney disease 4 with or without polycystic liver disease; PKD4, hepatic fibrosis, congenital, Gaucher disease, type I, type II, perinatal, lethal, prion protein; PRNP, lysosomal acid lipase deficiency, Wolman disease, inflammatory bowel disease (Crohn disease) 1; IBD1, regional enteritis, protracted neurovisceral, glucosidase, beta, acid; GBA glucocerebrosidase pseudogene; GBAP, protein-tyrosine kinase SYK; SYK, spleen focus forming virus proviral integration oncogene SPI1; SPI1, apolipoprotein e; APOE, SICKLE CELL ANEMIA, telangiectasia, hereditary hemorrhagic, type 1; HHT1, lymphoproliferative syndrome, X-linked, 1; XLP1, Letterer-Siwe disease, cerebral arteriopathy, autosomal recessive, with subcortical infarcts and leukoencephalopathy; CARASIL, Farber lipogranulomatosis; FRBRL, hemophilia A; HEMA, erythroid-associated factor; ERAF, ceroid storage disease, nucleotide-binding oligomerization domain protein 2; NOD2, porphyria, congenital erythropoietic, histiocytosis-lymphadenopathy plus syndrome, diabetes mellitus, insulin-dependent; IDDM, osteopetrosis, autosomal recessive 1; OPTB1, glycogen storage disease Ib; GSD1B, Ia, Tangier disease; TGD, cytotoxic t lymphocyte-associated 4; CTLA4, cytotoxic t lymphocyte-associated 4, soluble isoform, right atrial isomerism; rai, polysplenia syndrome, autoimmune disease, multisystem, with facial dysmorphism; ADMFD, Kaposi sarcoma, mucopolysaccharidosis, type IVA; MPS4A, zinc finger gene in autoimmune thyroid disease 1; ZFAT1, hemoglobin-beta locus; HBB, maple syrup urine disease; MSUD, maple syrup urine disease, classic, polycystic liver disease 2 with or without kidney cysts; PCLD2, beta-glucuronidase; GUSB, neutrophilic dermatosis, acute febrile, Gillessen-Kaesbach-Nishimura syndrome; GIKANIS, hemophagocytic lymphohistiocytosis, familial, 1; FHL1, NPC1 gene; NPC1, dehydrated hereditary stomatocytosis 1 with or without pseudohyperkalemia and/or perinatal edema; DHS1, sphingomyelin phosphodiesterase 1, acid lysosomal; SMPD1, hemochromatosis, type 1; HFE1, Gaucher disease, type III, IIIA, glycogen storage disease III; GSD3, glycogen storage disease Ma, Mb, Inc, IIId, IXa1, IXa2, VI, reticular dysgenesis, nk2 homeobox 5; NKX2-5, rh-null, regulator type; RHNR, RH-MOD, leucine zipper protein 6; LUZP6, interleukin 10; IL10, glycoprotein storage disease, granulomatous disease, chronic, X-linked; CDGX, cytochrome b-positive granulomatous disease, chronic, von Willebrand disease, type 2; VWD2, type 2A, type 2B, type 2M, type 2N, VHL gene; VHL, mast cell disease, urticaria pigmentosa, Hurler syndrome, hypermanganesemia with dystonia 1; HMNDYT1, chitinase 1; CHIT1, interleukin 6; IL6, Creutzfeldt-Jakob disease; CJD, polycystic kidney disease 1 with or without polycystic liver disease; PKD1, Alzheimer disease; AD, programmed cell death 1; PDCD1, hemochromatosis, neonatal, severe combined immunodeficiency, autosomal recessive, t cell-negative, b cell-negative, NK cell-negative, due to adenosine deaminase deficiency; SCID due to ADA deficiency, lymphoblastic leukemia, acute, with lymphomatous features; LALL, phosphatase, acid, type 5, tartrate-resistant; ACP5, serum amyloid A1; SAA1, adenosine A2A receptor; ADORA2A, leprosy, susceptibility to, 1; LPRS1, arrestin, beta, 1; ARRB1, CLN3 gene; CLN3, Sandhoff disease, Tay-Sachs disease; TSD, hexosaminidase A deficiency, adult type, included; GM2-gangliosidosis, Chanarin-Dorfman syndrome; CDS, Danon disease, Darier-White disease; DAR, Darier disease, Menkes disease, Charcot-Marie-tooth disease; CMTD, Parkinson disease PARK, melanocortin 5 receptor; MC5R, protein kinase C, delta; PRKCD, Huntington disease; HD, autoimmune lymphoproliferative syndrome, type III; ALPS3, leukemia, chronic myeloid; CML, tumor necrosis factor ligand superfamily, member 13B; TNFSF13B, prosaposin; PSAP, saposin A, lysosome-associated membrane protein 2; LAMP2, G protein-coupled receptor 183; GPR183, solute carrier family 37 (glucose-6-phosphate transporter), MEMBER 4; SLC37A4, micro ma 155; MIR155, immunity-related GTPase family, M; IRGM, chromosome 5q deletion syndrome megakaryocytes, unilobular nucleated, scavenger receptor class B, member 2; SCARB2, amyloidosis, familial visceral, transcription factor 21; TCF21, retinoic acid receptor, gamma; RARG, Omenn syndrome, Hirschsprung disease 1; HSCR1, legg-calve-pertes disease; LCPD, ceroid lipofuscinosis, neuronal, 3; CLN3, polycystic kidney disease 3, 2; PKD3, PKD2, infantile sialic acid storage disease; ISSD, adrenoleukodystrophy; ALD, adrenomyeloneuropathy, AMN, central core disease of muscle; CCD, minicore myopathy; CMT2A1, liver glycogenosis, X-linked, type II; XLG2, dementia, Lewy body; DLB, hemoglobin H disease; HBH, peroxisome biogenesis disorder 1B; PBD1B, pulmonary disease, chronic obstructive; COPD, erythrocytosis, familial, 2; ECYT2, apolipoprotein A-I; APOA1, APOA1 deficiency, myeloid zinc finger gene 1; MZF1, estrogen receptor 1; ESR1, HDL cholesterol, SH2 domain protein 2A; SH2D2A, severe combined immunodeficiency, X-linked; SCIDX1, Schimke immunoosseous dysplasia; SIOD, interferon-gamma receptor 1; IFNGR1, Glanzmann thrombasthenia; GT, galactosialidosis; GSL, solute carrier family 40 (iron-regulated transporter), member 1; SLC40A1, catenin, beta-1; CTNNB1, complement component 3; C3, C3a, C3b, C3c, C3 d, acylation-stimulating protein, ASP, NK2 homeobox 3; NKX2-3, CD44 antigen; CD44, NLR family, caspase recruitment domain-containing 4; NLRC4, HFE gene; HFE, APC gene; APC, ceroid lipofuscinosis, neuronal, 10; CLN10, peroxisome proliferator-activated receptor-gamma; PPARG, caspase recruitment domain-containing protein 8; CARDS, major histocompatibility complex, class II, transactivator; MHC2TA, sphingosine-1-phosphate receptor 1; S1PR1, selenoprotein N; SELENON, interleukin 18; IL18, breakpoint cluster region; signal transducer and activator of transcription 3; STAT3, protein-tyrosine phosphatase, nonreceptor-type, 11; PTPN11, protein tyrosine phosphatase, nonreceptor-type, 22; PTPN22, vitamin D receptor; VDR, solute carrier family 29 (nucleoside transporter), member 3: SLC29A3, RAR-related orphan receptor C; RORC, chemokine, cxc motif, ligand 12; CXCL12, neuraminidase deficiency, sialidosis, type I, ring finger protein 213; RNF213, solute carrier family 11 (proton-coupled divalent metal ion transporter), member 1; SLC11A1, integrin, beta-2; ITGB2, leukocyte-associated antigens CD18/11A, CD18/11B, CD18/11C, V-KIT Hardy-Zuckerman 4 feline sarcoma viral oncogene homolog; KIT, methyl-CpG-binding protein 2; MECP2, familial mediterranean fever gene; MEFV, calpain 5; CAPN5, sorbin and SH3-domains containing protein 1; SORBS1, inhibitor of kappa light polypeptide gene enhancer in B cells, kinase of, gamma; IKBKG, caspase 3, apoptosis-related cysteine protease; CASP3, tumor necrosis factor receptor superfamily, member 11A; TNFRSF11A, placental growth factor; PGF, KIT ligand; KITLG, interleukin 18 receptor 1; IL18R1, autoimmune regulator; AIRE, chemokine, cc motif, ligand 2; CCL2, coronary artery disease (based on OMIM database: http://omim.org/search/?index=entry&start=1&limit=10&sort=score+desc %2C+prefix_sort+desc&search=spleen+disease).

Specifically, the lung-associated associated disease, for which the delivery system of the invention will be useful, is selected from the following list and/or relates to disfunction in the following genes Cystic fibrosis; CF, pulmonary disease, chronic obstructive; COPD, transforming growth factor, beta-1; TGFB1, pulmonary venoocclusive disease 1, autosomal dominant; PVOD1, alpha-1-antitrypsin deficiency; A1ATAD, pulmonary venoocclusive disease 2, autosomal recessive; PVOD2, surfactant metabolism dysfunction, pulmonary, 2; SMDP2, pulmonary hypertension, primary; PPH, lung cancer, alveolar cell carcinoma, including: adenocarcinoma of lung, non-small cell lung cancer, lung cancer, surfactant metabolism dysfunction, pulmonary, 1; SMDP1, surfactant, pulmonary-associated protein c; SFPC, surfactant metabolism dysfunction, pulmonary, 3; SMDP3, bone morphogenetic protein receptor, type II; BMPR2, Mowat-Wilson Syndrome; MOWS, Noonan syndrome 1; NS1, Pterygium Colli syndrome, pulmonary fibrosis, idiopathic; IPF, Hamman-Rich disease, Niemann-Pick Disease, surfactant, pulmonary-associated protein A1; SFTPA1, interferon-related developmental regulator 1; IFRD1, telangiectasia, hereditary hemorrhagic, type 1; HHT1, serpin peptidase inhibitor, CLADE A, MEMBER 1; SERPINA1, dyskeratosis congenita, autosomal dominant 1; DKCA1, surfactant, pulmonary-associated protein B; SFTPB, microphthalmia, syndromic 9; MCOPS9, lymphoma, Hodgkin, classic; CHL, pulmonary fibrosis and/or bone marrow failure, telomere-related, 1; PFBMFT1, pulmonary hypertension, primary, 4; pph4, hedgehog-interacting protein; HHIP, surfactant metabolism dysfunction, pulmonary, 5; SMDP5, cystic disease of lung, asthma, total anomalous pulmonary venous return 1; TAPVR1, sodium channel, nonvoltage-gated 1, alpha subunit; SCNN1A, tetralogy of fallot; TOF, sickle cell anemia, NK2 homeobox 1; NKX2-1, Parkinson disease; PD, fibroblast growth factor 10; FGF10, Keutel syndrome; KTLS, IgE responsiveness, atopic; IGER, pulmonic stenosis, aplasia of lacrimal and salivary glands; ALSG, cirrhosis, jagged 1; JAG1, interstitial pneumonitis, desquamative, familial; DIP, telangiectasia, hereditary hemorrhagic, type 2; HHT2, pulmonary arterial hypertension, granulomatous disease, chronic, X-linked; CDGX, tumor protein p53; TP53, Gaucher disease, type I, elastin; ELN, sphingomyelin phosphodiesterase 1, acid lysosomal; SMPD1, Fabry disease, pulmonic stenosis and deafness, major histocompatibility complex, class I, B; HLA-B, neurofibromatosis, type I; NF1, rheumatoid arthritis; RA, Noonan syndrome 8; NS8, Hermansky-Pudlak syndrome 1; HPS1, TSC1 gene; TSC1, Young syndrome, V-KI-RAS2 Kirsten RAT sarcoma viral oncogene homolog; KRAS, velocardiofacial syndrome, surfactant metabolism dysfunction, pulmonary, 4; SMDP4, Williams-Beuren syndrome; WBS, mesothelioma, malignant; MESOM, epidermal growth factor receptor; EGFR, surfactant, pulmonary-associated protein D; SFTPD, Marfan syndrome; MFS, glucosidase, beta, acid; GBA, protein-tyrosine phosphatase, nonreceptor-type, 11; PTPN11, Alagille syndrome 1; ALGS1, atrial septal defect 1; ASD1, thyroid dyshormonogenesis 2A; TDH2A, Costello syndrome; CSTLO, major histocompatibility complex, class II, DR beta-1; HLA-DRB1, Smith-Lemli-Opitz syndrome; SLOS, arthrogryposis, distal, type 5; DA5, lymphangioleiomyomatosis; LAM, glycogen storage disease, GSD, supravalvular aortic stenosis; SVAS, heart defects, congenital, and other congenital anomalies; HDCA, telomerase ma component; TERC, right atrial isomerism; RAI, interstitial lung and liver disease; ILLD, activin A receptor, type II-like 1; ACVRL1, facioscapulohumeral muscular dystrophy 1; FSHD1, major histocompatibility complex, class II, DP beta-1; HLA-DPB1, Hermansky-Pudlak Syndrome 5; HPS5, breast cancer, central hypoventilation syndrome, congenital; CCHS, neurofibromin 1; NF1, severe combined immunodeficiency, lysosomal acid lipase deficiency, endothelin receptor, type B; EDNRB, matrix metalloproteinase 1; MMP1, tuberous sclerosis 1; TSC1, signal transducer and activator of transcription 3; STAT3, caveolin 1; CAV1, lung cancer susceptibility 1; LNCR1, Li-Fraumeni syndrome; LFS, pleuropulmonary blastoma; PPB, prostaglandin-endoperoxide synthase 2; PTGS2, hypertrophic osteoarthropathy, primary, autosomal recessive, 1; PHOAR1, cutis laxa, autosomal dominant 1; ADCL1, alpha-2-macroglobulin; A2M, dyskeratosis congenita, autosomal dominant 3; DKCA3, bronchiectasis with or without elevated sweat chloride 1; BESC1, patent ductus arteriosus 1; PDA1, muscleblind-like splicing regulator 1; MBNL1, telomerase reverse transcriptase; TERT, heterotaxy, visceral, 1, X-linked; HTX1, Birt-Hogg-Dube syndrome; BHD, lectin, mannose-binding, soluble, 2; MBL2, nephrotic syndrome, type 1; NPHS1, spinal muscular atrophy, type I; SMA1, phosphatase and tensin homolog; PTEN, Leopard syndrome 1; LPRD1, matrix metalloproteinase 9; MMP9, choreoathetosis and congenital hypothyroidism with or without pulmonary dysfunction; CAHTP, dyskeratosis congenita, X-linked; DKCX, Hoyeraal-Hreidarsson syndrome, toll-like receptor 4; TLR4, cutis laxa, autosomal recessive, type IC; ARCL1C, pulmonary function, immunoglobulin a deficiency 1; IGAD, lung agenesis, nitric oxide synthase 3; NOS3, interleukin 10; IL10, Letterer-Siwe disease, tuberous sclerosis 2; TSC2, chemokine, cc motif, ligand 2; CCL2, solute carrier family 6 (neurotransmitter transporter, serotonin), member 4; SLC6A4, lung cancer susceptibility 4; LNCR4, serpin peptidase inhibitor, clade A, member 3; SERPINA3, mucin 5, subtype B, tracheobronchial; MUCSB, amyotrophic lateral sclerosis; ALS, interleukin 13; IL13, dermatitis, atopic; ATOD, bpi fold-containing protein, family a, member 1; BPIFA1, Farber lipogranulomatosis; FRBRL, vascular endothelial growth factor A; VEGFA, Holt-Oram syndrome; HOS, granulomatous disease, chronic, autosomal recessive, cytochrome b-positive, type II; CDG2, minicore myopathy with external ophthalmoplegia, diaphragmatic hernia, DIH, cutis laxa, autosomal recessive, type IA; ARCL1A, chemokine, CC motif, receptor 5; CCR5, fibrillin 1; FBN1, FAS ligand; FASLG, interleukin 17A; IL17A, complement hyperactivation, angiopathic thrombosis, and protein-losing enteropathy; CHAPLE, brain small vessel disease with or without ocular anomalies; BSVD, presenilin 2; PSEN2, collagen, type i, alpha-1; COL1A1, bleomycin hydrolase; BLMH, forkhead box O3A; FOXO3A), ciliary dyskinesia, primary, 33; CILD33 (secretoglobin, family 1A, member 1; SCGB1A1) ataxia-telangiectasia; AT, pulmonary alveolar proteinosis, acquired, Loeys-Dietz syndrome 2; LDS2, angiotensin I-converting enzyme 2; ACE2, delta-like 4; DLL4, mitral valve prolapse 1; MVP1, Waardenburg syndrome, type 4A; WS4A, pulmonary hypertension, primary, 2; PPH2, ciliary dyskinesia, primary, 1; CILD1, rigid spine muscular dystrophy 1; RSMD1, pulmonary fibrosis and/or bone marrow failure, telomere-related, 4; PFBMFT4, Von Hippel-Lindau syndrome; VHL, pulmonary fibrosis and/or bone marrow failure, telomere-related, 3; PFBMFT3 (based on OMIM database: https://www.omim.org/search/?index=entry&sort=score+desc %2C+prefix_sort+desc&start=1&limit=10&search=lung+genetic+disease).

Specifically, the heart-related disease, for which the delivery system of the invention will be useful, manifest itself in high blood pressure, heart attacks, heart failure, and stroke and transient ischemic attack (TIA) and is associated with disfunction of one or combination of the following genes (see, e.g. WO2015089462A1): IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCGS (ATP-binding cassette, sub-family G (WHITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), VWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member 11b), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C-X-C motif) receptor 4), PROC (protein C (inactivator of coagulation factors Va and VIIIa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomerase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CABIN1 (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IL17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, A1 polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member 1B), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac1)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L1), PCNA (proliferating cell nuclear antigen), IGF2 (insulin-like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALCA (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PECAM1 (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-.beta.-methyltransferase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (B321 placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHC1 (SHC (Src homology 2 domain containing) transforming protein 1), LRP1 (low density lipoprotein receptor-related protein 1), SOCS3 (suppressor of cytokine signaling 3), ADH1B (alcohol dehydrogenase 1 B (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor H1), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQO1 (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCHE (butyrylcholinesterase), LIPE (lipase, hormone-sensitive), PRDXS (peroxiredoxin 5), ADORA1 (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CHGA (chromogranin A (parathyroid secretory protein 1)), IAPP (islet amyloid polypeptide), RHO (rhodopsin), ENPP1 (ectonucleotide pyrophosphatase/phosphodiesterase 1), PTHLH (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISH (cytokine inducible SH2-containing protein), GAST (gastrin), MYOC (myocilin, trabecular meshwork inducible glucocorticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), HSF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTH (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOH (apolipoprotein H (beta-2-glycoprotein I)), S100A8 (S100 calcium binding protein A8), IL11 (interleukin 11), ALOX15 (arachidonate 15-lipoxygenase), FBLN1 (fibulin 1), NR1H3 (nuclear receptor subfamily 1, group H, member 3), SCD (stearoyl-CoA desaturase (delta-9-desaturase)), GIP (gastric inhibitory polypeptide), CHGB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), HSD11B2 (hydroxysteroid (11-beta) dehydrogenase 2), CALCRL (calcitonin receptor-like), GALNT2 (UDP-N-acetyl-alpha-D-galactosamine:polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adenovirus E1B 19 kDa interacting protein 3), GCKR (glucokinase (hexokinase 4) regulator), S100A12 (S100 calcium binding protein A12), PADI4 (peptidyl arginine deiminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), IDDM2 (insulin-dependent diabetes mellitus 2), RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-1(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), KHK (ketohexokinase (fructokinase)), SAT1 (spermidine/spermine N1-acetyltransferase 1), GGH (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RHOB (ras homolog gene family, member B), LY96 (lymphocyte antigen 96), FOXO1 (forkhead box O1), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPR1 (nuclear protein, transcriptional regulator, 1), 15-Sep(15 kDa selenoprotein), CILP2 (cartilage intermediate layer protein 2), TERC (telomerase RNA component), GGT2 (gamma-glutamyltransferase 2), MT-CO1 (mitochondrially encoded cytochrome c oxidase I), and UOX (urate oxidase, pseudogene), Pon1 (paraoxonase 1), LDLR (LDL receptor), ApoE (Apolipoprotein E), Apo B-100 (Apolipoprotein B-100), ApoA (Apolipoprotein(a)), ApoA1 (Apolipoprotein A1), CBS (Cystathione B-synthase), Glycoprotein IIb/IIb, MTHRF (5,10-methylenetetrahydrofolate reductase (NADPH), Cacna1C, Sod1, Pten, Ppar(alpha), Apo E, Leptin, and combinations thereof.

Specifically, the kidney-associated disease, for which the delivery system of the invention will be useful, is selected from (see, e.g. U.S. Pat. No. 6,277,574B1): Bartter's syndrome, Gitelman syndrome, nephrolithiasis. renal amyloidosis, hypertension; primary aldosteronism; Addison's disease; renal failure; glomerulonephritis; chronic glomerulonephritis: tubulointerstitial nephritis; cystic disorders of the kidney and dysplastic malformations such as polycystic disease, renal dysplasias, and cortical or medullary cysts; inherited polycystic renal diseases (PRD), such as recessive and autosomal dominant PRD; medullary cystic disease; medullary sponge kidney and tubular dysplasia; Alport's syndrome; non-renal cancers which affect renal physiology, such as bronchogenic tumors of the lungs or tumors of the basal region of the brain; multiple myeloma; adenocarcinomas of the kidney; metastatic renal carcinoma; in addition, nephrotoxic disorders include any functional or morphologic change in the kidney produced by any pharmaceutical, chemical, or biological agent that is ingested, injected, inhaled, or absorbed. Affected genes belong to the group consisting of uromodulin, NKCC2 (bumetanide-sensitive Na—K—Cl cotransporter 2), NCCT (thiazine-sensitive Na—Cl cotransporter), aldolase B, ROMK1 (inwardly-rectifying voltage-gated K channel), ATP1G1 (Na—K ATPase gamma subunit), PDZK1 (PDZ domain-containing protein), NPT-1 (Na-dependent phosphate cotransporter), calbindin, kininogen, and CIC-Kb (chloride channel).

Presented system of antigen-defined EV-based delivery of DNA modifying enzymes in vivo utilizes EV-samples collected and processed in serum-free conditions. This is an important technological aspect for future clinical applications in human patients. Moreover, the data indicate that the serum-free EVs contain higher mRNA levels for the designer nucleases (ZFN, TALEN, CRISPR/Cas9), in comparison to serum-containing EVs. The population of stem cell-EVs which we propose as a delivery tool, stains with an RNA-selective dye (more than 10%) and presents the surface antigens: CD90, CD105, CD147, CD309 (more than 20% in RNA-positive fraction), and has no expression of CD45 (CD90+/CD105+/CD147+/CD309+/CD45−).

### BRIEF DESCRIPTION OF THE DRAWINGS

FIG. 1 presents the characterization of hUC-MSC cell lines stably expressing TALENs, ZFNs and the CRISPR/Cas9. Genetic modification was obtained by nucleofection of plasmid DNA containing designer nucleases, followed by puromycin selection for 6 days. A. Analysis of genomic DNA (gDNA) of modified cells. Schematic representation of nucleases sequences. Arrows illustrate primers binding sites for PCR-based genotyping. For each nuclease two reactions were performed. B. Relative gene expression levels in hUC-MSC-DN. Genes related to apoptosis (BAX, BCL2), mesoderm (COL1A1, MSX1), ectoderm (PAX6), endoderm (SOX17), pluripotency (NANOG, SOX2) proliferation status (c-MYC) and nucleases were studied. Data were normalized to the expression of an endogenous gene (β2-microglobulin) and compered to results obtained from wild type hUC-MSC. Value equal to 1 represents gene expression in hUC-MSC-WT. C. Protein expression of DN in genetically modified hUC-MSC. Antibodies directed toward HA-epitope or His-Tag were used to detect expression of designer nucleases in UC-MSC-DN. Anti-β-actin antibody was used as control.

FIG. 2 presents the isolation and characterization of extracellular vesicles (EVs) derived from UC-MSCs expressing designer nucleases (DN-EVs). A. Size distribution of DN-EVs analyzed by the NanoSight analyzer. B. Visualization of DN-EVs using Transmission Electron Microscopy (TEM). Representative TEM pictures are shown with a scale bar of 500 nm and 200 nm. C. Surface antigen analysis on DN-EVs. The particles were stained with selected antibodies labeled with fluorescent markers and analyzed on a A50-Micro Flow Cytometer (Apogee Flow Systems). MALS—Medium Angle Light Scatter. D. Analysis of mRNA levels for designer nucleases (ZFN, TALEN and gRNA/Cas9) in DN-EVs using real time qPCR technique and calculated with the ΔΔCt method. Expression of β2-microglobulin was used as endogenous control and levels of mRNA expression were calibrated with WT-EVs (level 1 on y axis).

FIG. 3 presents the comparison of transcript levels for designer nucleases (DNs) in extracellular vesicles (EVs) collected in serum-containing or serum-free medium. DN-EVs were harvested from conditioned media from UC-MSC-DNs grown in DMEM/F12 supplemented with 10% fetal bovine serum (FBS) or the medium was replaced with DMEM/F12 containing 0.5% bovine serum albumin (BSA) for 48 h, before EVs collection. Analysis of transcript levels was performed with real time qPCR method, using β2-microglobulin as endogenous control and FBS-EVs as calibrator (level 1 on y axis).

FIG. 4 presents the EGFP knockout in EGFP-expressing cell lines mediated by DN-EVs. A. Co-culture system of DN-expressing cell lines with HEK-EGFP and UC-MSC-EGFP cell lines. DN-EVs-producer cells and target cells were cultured in Boyden chambers separated by a mesh filter with 0.4 μm pores for 10 days. EGFP knockout was measured by flow cytometry. B. Transfer of DN-EVs to EGFP-expressing cell lines. Cells were treated up to five times with DN-EVs in a dose of 1 μg of EVs/10⁴ cells. EGFP knockout was measured at day 5 after the last MVs transfer by flow cytometry. C. Transcript levels of ZFNs in HEK-293T-EGFP and UC-MSC-EGFP cells after incubation with ZFN-EVs at indicated time points (2, 6, 16 and 26 h).

FIG. 5 presents the results of analysis of EGFP gene knockout in various tissues of NOD/SCID-EGFP mice upon injection of UC-MSC-EVs containing designer nucleases (DN-EVs). Mice were transplanted with DN-EVs either intravenously or intraperitoneally and were sacrificed 7 days post-injection. Cells from various organs were isolated and EGFP knockout was measured by flow cytometry. Results are shown as a mean and standard deviation from two experiments, after background subtraction from PBS-injected animals. A. EGFP knockout after single intravenous injection of MVs containing different type of nucleases in a dose of 20 μg. B. EGFP knockout measured after single (1×) or triple (3×) administration of CRISPR/Cas9-EVs in a dose of 20 μg or 60 μg. C. EGFP knockout measured after single intraperitoneal injection of 20 μg EVs containing various types of nucleases.

### EXAMPLES

### Example 1: Generation of Stem Cell Lines Stably Producing DNA Modifying Enzymes

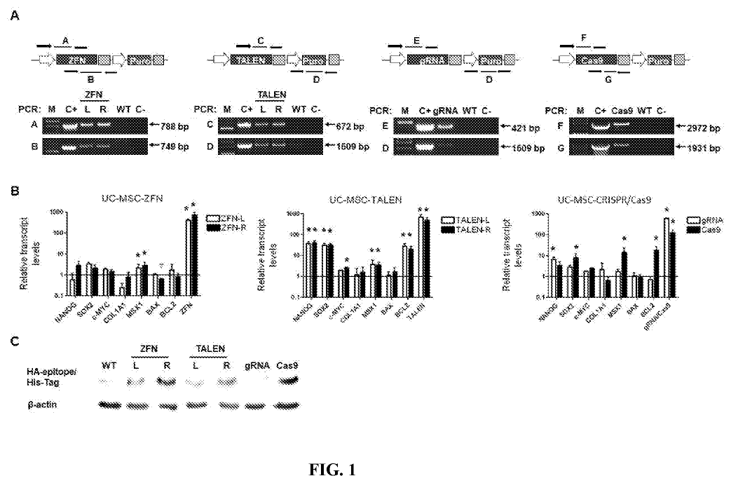

Designer nucleases (DN) producer cell lines were generated by transfection of umbilical cord-derived mesenchymal stem cells (UC-MSs) with vectors expressing designer nucleases (ZFN, TALEN, RGEN) using the Neon Transfection System (Invitrogen) and the following parameters: 1400 V, pulse width: 10 ms and pulse number: 3. Cells were cultured in DMEM/F12 medium (Sigma-Aldrich) containing 10% fetal bovine serum (FBS; Gibco), 10,000 U/ml penicillin, 10,000 μg/ml streptomycin (P/S; Gibco) in cell culture flasks (75 cm², 175 cm²) or cell culture dishes (10 cm in diameter; all from Becton Dickinson). To enrich for gene targeted cells, selection with puromycin at 0.8 μg/mL for 6 days was performed. Insertion of DN sequences into genomic DNA was confirmed by PCR-based genotyping (FIG. 1A). First, genomic DNA (gDNA) was isolated using GeneMATRIX Cell Culture DNA Purification Kit (EURx). Reactions were carried out with the OptiTaq PCR Master Mix (EURx), specific primer pairs (listed in Table 1) at 0.4 mM final concentration and 60 ng of gDNA per 20 μl reaction. After initial denaturation at 95° C. for 5 min, 30 reaction cycles were performed, containing the following steps: denaturation at 95° C. for 20 sec., primers annealing at 66° C. for 30 sec. and extension at 72° C. for 45 sec., followed by final extension at 72° C. for 7 min. Reaction products were visualized in 1% agarose gel after electrophoresis.

Additionally, expression of the nucleases at mRNA and protein levels was confirmed by RT-qPCR (FIG. 1B) and Western blot techniques (FIG. 1C), respectively. RT-qPCR was performed after total RNA isolation using the GeneMATRIX Universal RNA Purification Kit (EURx). To remove any residual traces of genomic DNA, an additional step with DNAse digestion (0.02 U/μl of TURBO™ DNase; Ambion) was included. cDNA was synthesized using the dART RT-PCR kit (EURx), according to the manufacturer's instruction, with 50 μM Oligo(dT)20 and 200 ng/μl random hexamers. Reactions were performed in the C1000 Touch™ Thermal Cycler (Bio-Rad) using the following parameters: 10 min at 25° C., followed by 40 min at 50° C., and 5 min at 85° C.

Quantitative Real-Time PCR—qPCR was conducted on the MicroAmp Optical 96-well plates with the SYBR Green PCR Master Mix combined with specific forward and reverse primers (at final concentration of 0.5 μM; listed in Table 2), 30 ng of cDNA per well and covered with the MicroAmp Optical Adhesive Film, using the 7500 Fast Real-Time PCR System (all from Applied Biosystems/Thermo Fisher Scientific). Relative gene expression level was calculated using the ΔΔCt method and B2M as endogenous control.

To perform Western blot analysis, samples were lysed in 100 μl of ice-cold lysis buffer composed of 50 mM Tris-HCl (pH 7.5), 150 mM NaCl, 0.1% Tritin X-100, 0.002% sodium azide (Sigma-Aldrich/Merck) with a cocktail of phosphatase inhibitors (Sigma-Aldrich). Samples were boiled at 95° C. for 5 min and separated by SDS-PAGE (Sodium Dodecyl Sulfate Polyacrylamide Gel Electrophoresis) 12% and transferred to polyvinylidene difluoride (PVDF) membranes. ZFN, TALEN and Cas9 expression was detected using antibodies directed against HA tag (#NB600-363; Novus Biologicals, dilution: 1:900) and His-taq (#2365; Cell Signaling; dilution: 1:1000), respectively. Control staining was performed using anti-β-actin (#sc-81178, Santa Cruz Biotechnology; dilution: 1:1000). Proteins were stained with HRP-conjugated goat anti-mouse IgG (Molecular Probes/Thermo Fisher Scientific; dilution 1:3000), goat anti-rabbit IgG (Molecular Probes; dilution 1:3000) secondary antibodies using the WestPico Chemiluminescence substrate (Thermo Fisher Scientific) and visualized by the MicroChemi (DNR Bio-Imaging Systems).

These results indicate simplicity and versatility of DN-packaging cell line generation, for EVs collection. Variable cell lines expressing single or multiple domains for various DNA-modifying enzymes can be created in a similar way.

TABLE 1
Sequences of primers used for PCR-based
genotyping of genetically modified UC-
MSCs stably expressing designer nucleases.

| | | | | Ampli- |
|---|---|---|---|---|
| | | | | con |
| | Detection | Primer | Primer | lengh |
| PCR | of: | forward | reverse | (bp) |
| A | Junction of | TTTGCCCTTT | GATAGGCAGGT | 788 |
| | EF1a promoter | TTGAGTTTGG | TGTAGCCGC | |
| | and ZFN | | | |
| B | Junction of | GAACTGATCG | TGGGCCTTCAC | 749 |
| | ZFN and TKpA | AAATCGCCCG | CCGAACTT | |
| C | Junction of | ATCAAGGAAA | TGGGCCTTCAC | 672 |
| | TALEN and | CCGGACGGAG | CCGAACTT | |
| | TKpA | | | |
| D | PGK -puro-pA | GGGGTTGGGG | GCCTGCTATTG | 1609 |
| | cassette | TTGCGCCT | TCTTCCCAA | |
| E | Junction of | ATCGATGCTA | GACTCGGTGCC | 421 |
| | U6 promoter | GCTGTACAAA | ACTTTTTCA | |
| | and gRNA | AAAGC | | |
| F | Junction of | TTTGCCCTTT | GTGATTTGGCG | 2972 |
| | EF1a promoter | TTGAGTTTGG | GGTTTCCAC | |
| | and Cas9 | | | |
| G | Junction of | AAAGTGCTTA | TGGGCCTTCAC | 1931 |
| | Cas9 and TKpA | CACGCTCGGA | CCGAACTT | |

TABLE 2
Primer sequences used for RT-qPCR.

| Gene name | | Sequence |
|---|---|---|
| B2M | F | AATGCGGCATCTTCA |
| (beta-2- | R | TGACTTTGTCACAGCCCAAGATA |
| microglobulin) | | |
| BAX | F | GTCTTTTTCCGAGTGGCAGC |
| | R | GTCCAATGTCCAGCCCATGA |
| BCL2 | F | TTGCTTTACGTGGCCTGTTTC |
| | R | GAAGACCCTGAAGGACAGCCAT |
| c-MYC | F | ATGTCCTGAGCAATCACCTATG |
| | R | AAGTTCTTTTATGCCCAAAGTCC |
| COL1Al | F | GGACACAGAGGTTTCAGTGGT |
| | R | GCACCATCATTTCCACGAGC |
| NANOG | F | ACCTCAGCTACAAACAGGTGAAG |
| | R | TTCTGCGTCACACCATTGCT |
| SOX2 | F | GGGAAAGTAGTTTGCTGCCTC |
| | R | CAGGCGAAGAATAATTTGGGGG |
| MSX1 | F | RT² qPCR Primer Assay |
| | | for Human MSX1: |
| | R | PPH02118F (Qiagen) |
| ZFN | F | GAACTGATCGAAATCGCCCG |
| | R | GATAGGCAGGTTGTAGCCGC |
| TALEN | F | ATCAAGGAAACCGGACGGAG |
| | R | ATCGTTGCATTTCATCTGCTTGG |
| gRNA | F | GTTTTAGAGCTAGAAATAGCAA |
| | R | GACTCGGTGCCACTTTTTCA |
| Cas9 | F | GGGGAACACAGACCGTCATT |
| | R | TTCGGTTCTTGCGACGTGTA |

### Example 2: Collection and Characterization of DN-EVs

For EVs collection, DN-producer stem cell lines were cultured to sub-confluency (80-90% of cell density) in standard conditions (DMEM/F12 supplemented with 10% FBS and P/S, as described in Example 1), then they were washed twice in PBS (2×5 ml in 75 cm² flask) and the medium was changed into serum-free medium (DMEM/F12 supplemented with 0.5% bovine serum albumin; BSA, Sigma-Aldrich and P/S) for 48 h. Conditioned media were collected and were used for EVs collection, using sequential ultracentrifugation method. First, supernatants were centrifuged at 2000×g for 20 min at 4° C. to remove remaining cells, debris and apoptotic bodies. Next, the supernatants were subjected to ultracentrifugation at 100 000×g for 70 min at 4° C. in 30 ml polypropylene ultracentrifuge tubes (PP Oak Ridge Tube, Thermo Fisher Scientific) in an ultracentrifuge Optima XPN-90 K using fixed angle rotor 50.2 TI (Beckman Coulter, Brea, Calif., USA). After ultracentrifugation, the supernatants were discarded and the EV-pellets were resuspended in 1-2 ml PBS, pooled from several tubes (6-8) into one ultracentrifuge tube and subjected to the second ultracentrifugation with the same parameters. The pellets were resuspended in 150-200 μl of PBS (Lonza) and were aliquoted for long-term storage at −80° C. Protein concentration in EV specimens was determined with the Bradford assay. Size distribution and concentration was analyzed using the NanoSight NS300 instrument (Malvern Panalytical, Malvern, UK).

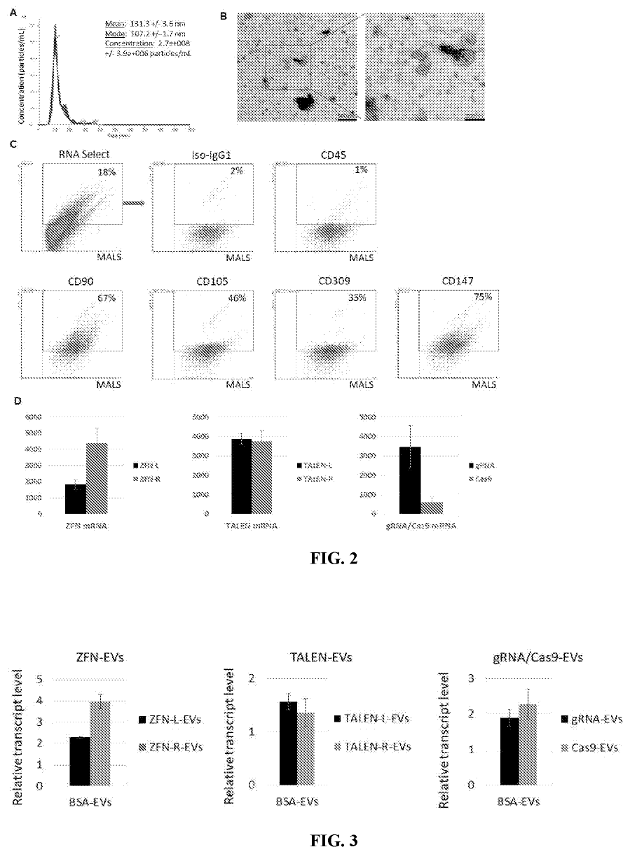

The determined mean size of EVs was in a range of 115-140 nm in diameter and did not differ significantly between DN-EV types. A representative histogram is shown in the FIG. 2A. EVs were also visualized by Transmission Electron Microscopy (TEM; FIG. 2B).

To characterize EVs phenotype, staining with antibodies against stem cell surface markers was performed. To ensure that only circular structures were analyzed, RNA-specific dye was used, since EV cargo comprises mainly small RNAs. The staining was performed as follows: the SYTO RNASelect green fluorescent cell stain (#S32703; Molecular Probes) was diluted in PBS (Lonza) according to the supplier's instruction and centrifuged for 20 min at 21 000×g at 4° C. to remove dye aggregates. EVs suspended in PBS (Lonza) were incubated with the RNA dye and selected antibodies (APC-labeled: CD45, CD105, CD309, CD147 from BioLegend or CD90-AlexaFluor 647-labeled from BD Pharmigen) for 15 min in the dark. The percentage of positive events was measured with the Apogee A50-Micro Flow Cytometer and calculated using the Apogee Histogram software (Apogee Flow Systems).

The DN-EVs samples contained at least 10% of RNA-positive events, were CD45-negative and displayed expression of selected stem cells surface markers in more than 20% (FIG. 2C, Table 3). The presence of DN transcripts and proteins were analyzed by RT-qPCR and Western blot methods, as described previously.

As the results indicated, mRNA levels were highly upregulated in EVs derived from genetically modified stem cells, in comparison to unmodified (wild type; WT) EVs (FIG. 2D). Proteins for ZFN, TALENs and. Cas9 could not be detected in EVs with the Western blot method (data not shown).

TABLE 3
Expression of stem cell-characteristic surface antigens
on DN-EVs measurer by A50-Micro Flow Cytometer.

| ANTIGEN | MEAN % | SD |
|---|---|---|
| CD45 | 0 | 01 |
| CD90 | 51 | 11.7 |
| CD105 | 34 | 6 |
| CD309 | 26 | 57 |
| CD147 | 65 | 4.2 |

These results demonstrate that EVs collected from genetically modified stem cell lines using the ultracentrifugation method or other techniques (centrifugation in sucrose gradient, or using OptiPrep or other density gradient media, or commercially available EV-isolation kits), contain high levels of mRNAs for DNA modifying enzymes (such as ZFN, TALEN, CRISPR/Cas9) and can be characterized as vesicles having more than 100 nm in diameter and expressing stem cells surface markers: CD90, CD105, CD309, CD147 (more than 20% in RNA-positive fraction) and are CD45-negative.

### Example 3: Comparison of DN Transcript Levels in EVs Collected from Serum-Containing or Serum-Free Medium

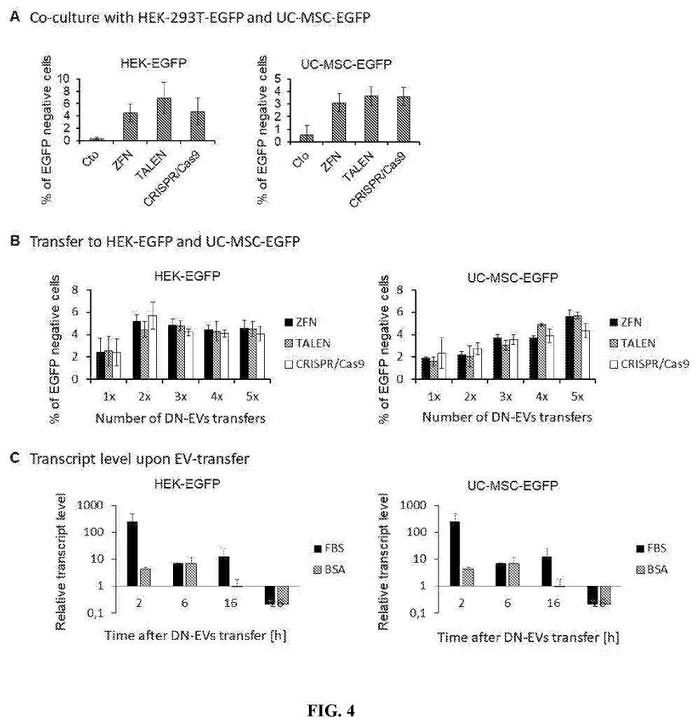

To compare efficacy of DN-packaging into EVs, DN-UC-MSCs were cultured either in standard conditions, in DMEM/F12 supplemented with 10% fetal bovine serum (FBS) and P/S, or the medium was replaced with DMEM/F12 containing 0.5% bovine serum albumin (BSA) and P/S for 48 h, to collect cell supernatants. EVs were harvested from conditioned media by sequential ultracentrifugation method, as described previously. The total RNA was isolated from DN-EV-specimens using the Total Exosome RNA & Protein Isolation Kit (Invitrogen) and was reverse transcribed to cDNA with the NG dART RT Kit (Eurx) according to the manufacturer's instruction. Transcript levels for DNs (ZFN, TALEN, gRNA/Cas9) were measured using the RT-qPCR technique and calculated with the ΔΔCt method using β2-microglobulin as endogenous control, as described previously (in the Example 1). Results indicate that DN-EVs collected in serum-free conditions contained higher transcript levels for DNs, in comparison to standard conditions, in the presence of serum (FIG. 3).

These results indicate that serum-free medium should be preferentially selected for EVs collection for enrichment of transcript levels for DNA modifying enzymes.

### Example 4: Activity of DNA Modifying Enzymes Transferred by Stem Cells-Derived EVs to Target Cells In Vitro

We tested activity of DNA modifying enzymes transferred by stem cells-derived EVs in a model system, in which EGFP target gene was disrupted by DNs in two cell types: HEK-293T-EGFP and primary hUC-MSC-EGFP cells. The decline in EGFP gene expression in target cells was measured by flow cytometry (BD LSR Fortessa). Two systems were used: i) the co-culture of a nuclease expressing MSC with a target cell line expressing EGFP gene using a transwell system; and ii) direct transfer of DN-EVs to EGFP-positive cell lines. In the latter option, a single and multiple DN-MVs treatments were performed. According to the results, up to 8% of EGFP gene knockout in the co-culture system (FIG. 4A) was obtained, as well as about 6% via direct transfer of DN-MVs (FIG. 4B). To confirm that the EGPF knockout was mediated by stem cell-derived DN-EVs, we measured DN transcript levels in the recipient cell lines after incubation with DN-EVs using RT-qPCR method, as described before (in the Example 1). In this study, the target cells were cultured in standard conditions (cell culture medium supplemented with 10% FBS) or cells were starved for 24 h (cell culture medium containing 0.5% BSA) before DN-EVs addition (1 μg EVs/10⁴ cells), to synchronize the cells in the cell cycle.

The results demonstrate that mRNA for DNs was detectable in the recipient cells particularly after 2 h upon DN-EVs addition (FIG. 4C). The EV uptake was more efficient in cells cultured in standard cell culture medium, which were not starved (FIG. 4C).

These results present an optimization step for selecting standardized conditions for DN-EVs transfer, to achieve in vitro genome editing in a cell line and in primary cells. Such conditions can be applied for the transfer of other DNA-modifying enzymes to variety of cell lines.

### Example 5: In Vivo Genome Editing Mediated by Stem Cells-Derived EVs Carrying DNA Modifying Enzymes

Stem cell-derived EVs containing DNs (ZFN, TALEN, CRISPR/Cas9 targeting the EGFP gene) were transplanted to NOD/SCID-EGFP transgenic mice in a dose of 20 μg. The EGFP knockout was measured 7 days post-injection in various organs. Two routes of MVs administration were tested, intravenous (into venous sinus) and intraperitoneal. To measure percentage of EGFP-negative cells, selected organs (spleen, lungs, heart, kidneys and liver) were collected from the animals, minced through 70 μm-pore filters to mechanically release cells. EGFP gene knockout was analyzed by flow cytometry using BD LSR Fortessa (BD Biosciences) and calculated after background subtraction from PBS-injected animals.

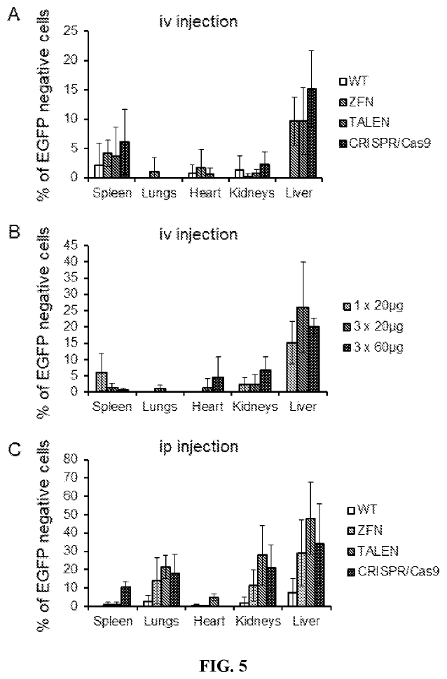

The results show, that single intravenous administration of DN-EVs resulted in 1 to 15% of EGFP-negative cells with the highest percentage in the liver (FIG. 5A). Multiple injections of DN-EVs (here: CRISPR/Cas9-EVs) increased the number of EGFP-negative cells up to 30% (FIG. 5B). Interestingly, intraperitoneal transplantation of DN-EVs resulted in higher EGFP-gene knockout rate in several organs (up to 50% in the liver; FIG. 5C).

### CONCLUSIONS

These results indicate that stem cell-derived EVs collected in serum-free conditions and characterized by the presence of stem cells surface markers: CD90/CD105/CD309/CD147+ and CD45-negative, constitute novel tool for efficient in vivo genome editing, particularly in the liver. This technology can be applied for treatment of variety genetic diseases in human patients.

Since this data showed enhanced gene targeting in the liver using this technology, liver-associated diseases can be of particular interest as potential targets, including: phenylketonuria (PAH, PKU1, QDPR, DHPR, PTS), amyloidosis (APOA1, APP, AAA, CVAP, AD1, GSN, FGA, LYZ, TTR, PALB); cirrhosis (RT18, T8, CIRH1A, NAIC, TEX292, KJAA1988); hypercholesterolemia (PCSK9), non-alcoholic liver disease (FAS), amyloid neuropathy (TTR, PALB); cystic fibrosis (CFTR, ABCC7, CF, MRP7); glycogen storage diseases (SLC2A2, GLUT2, G6PC, G6PT, G6PT1, GAA, LAMP2, LAMPB, AGL, GDE, GBE1, GYS2, PYGL, PFKM); polycystic kidney and hepatic disease (FCYT, PKHD1, ARPKD, PKD1, PKD2, PKD4, PKDTS, PRKCSH, G19P1, PCLD, SEC63); hepatic failure (SCOD1, SCO1), hepatic lipase deficiency (LIPC), medullary cystic kidney disease (UMOD, HNFJ, FJHN, MCKD2, ADMCKD2); hepatoblastoma, cancer and carcinomas (CTNNB1, PDGFRL, PDGRL, PRLTS, AXIN1, AXFN, CTNNB1, TP53, P53, LFS1, IGF2R, MPRI, MET, CASP8, MCH5); hepatic adenoma, (TCF1, HNF1A, MODY3) and others (PCSK9; Hmgcr; SERPINA1; ApoB; LDL).

This technology can be applied in basic science and importantly, in clinical therapies in the future, where both precision and safety of the treatment have to be considered. Antigen defined EVs (CD90+/CD105+/CD147+/CD309+/CD45−) can be collected from various populations of stem cells, including mesenchymal stem cells and induced pluripotent stem cells. Since the method of the generation of stem cell lines stably producing DNA modifying enzymes is basically the same for any enzyme, different types of DNA modifying enzymes can be delivered to target cells with this invention, including hybrid nucleases (ZFN, TALEN, CRISPR/Cas9, meganucleases), recombinases (Flp, Cre, Dre, KD, B2, B3, RadA, RAD51, RecA, Dmc1, etc.), integrases (λ, HK022, HP1, ϕC31, Bxb1, R4), resolvases/invertases (γδ, ParA, Tn3, Gin), transposases and others.

This technology can be used to create gene knockouts, knock-ins and to achieve gene correction, if additional DNA donor is provided (ss/sdDNA oligos, plasmid DNA, viral vectors).

Therapeutic gene knockout can be applied to treat HIV-infected patients (target genes: e.g. CCR5, CXCR4), leukemic patients (e.g. TRAC, CD52), solid tumors (e.g. PDCD1), HPV-induced cervical precancerous lesions (HPV E7), hypercholesterolemia (PCSK9), non-alcoholic liver disease (FAS), Duchenne muscular dystrophy (DMD), among others.

If additional DNA donor is provided for gene correction, variety of human diseases can be treated with this technology (human genetic diseases listed e.g. in the eDGAR database: http://edgar.biocomp.unibo.it/cgi-bin/gene_disease_db/main_table.py; 20). The surface of EVs can be either unmodified, carrying natural panel of stem cell specific antigens, or can be genetically or chemically modified to display additional receptors, ligands or other surface molecules, to increase targeting a specific tissue or a desired cell type.

1. In vivo delivery system of the DNA modifying enzymes comprising the stem cells-derived extracellular vesicles collected in serum-free conditions, characterized in that the said extracellular vesicles are the population of extracellular vesicles defined by the expression of surface markers.

2. The delivery system according to claim 1, wherein the extracellular vesicles are microvesicles.

3. The delivery system according to claim 1, wherein the population of extracellular vesicles is defined by the expression of surface markers: CD90, CD105, CD147, CD309 and lack of expression CD45.

4. The delivery system according to claim 1, wherein at least 10% of the extracellular vesicle's population contains mRNA.

5. The delivery system according to claim 4, wherein the mRNA is selected from the mRNA of the hybrid nucleases, recombinases, integrase, resolvases/invertases and transposases.

6. The delivery system according to claim 5, wherein the hybrid nucleases are selected from the Zinc Finger Nucleases (ZFN), Transcription Activator-Like Effector Nucleases (TALEN) and the CRISPR/Cas9 and the meganucleases.

7. The delivery system according to claim 5, wherein the recombinases are selected from the Flp, Cre, Dre, KD, B2, B3, RadA, RAD51, RecA and Dmc1.

8. The delivery system according to claim 5, wherein the integrases are selected from the λ, HK022, HP1, ϕC31, Bxb1 and R4.

9. The delivery system according to claim 5, wherein the resolvases/invertases are selected from the γδ, ParA, Tn3 and Gin.

10. The delivery system according to claim 4, wherein at least 20% of the RNA-positive population presents the surface antigens: CD90, CD105, CD147, CD309 and has no expression of CD45.

11. The delivery system according to claim 1, wherein the stem cell is selected from the mesenchymal stem cell and pluripotent stem cell.

12. The delivery system according to claim 11, wherein the mesenchymal stem cell is a umbilical cord-derived mesenchymal stem cell.

13. The delivery system according to claim 11, wherein the pluripotent stem cell is the induced pluripotent stem cell.

14. The delivery system as defined in any of previous claims for use in a treatment of a genetic disease and/or disorder.

15. The use according to claim 14, wherein the genetic disease and/or disorder is a mammalian genetic disease and/or disorder.

16. The use according to claim 14, wherein a genetic disease and/or disorder is selected from the liver-associated diseases, spleen-associated diseases, lung-associated diseases, heart-associated diseases and kidney-associated diseases.

17. The use according to claim 15, wherein the liver-associated disease is selected from the following list and/or relates to disfunction in the following genes: fatty liver disease, nonalcoholic, NAFLD, polycystic kidney disease with or without polycystic liver disease; PKD, glycogen storage disease, GSD, thyroid dyshormonogenesis 4; TDH4, hepatocellular carcinoma, hepatoblastoma, Niemann-Pick disease, NPD, alpha-1-antitrypsin deficiency; A1ATD, mitochondrial DNA depletion syndrome 4A (ALPERS type); MTDPS4A, maple syrup urine disease; MSUD, pyruvate kinase, liver and red blood cell; PKLR, alkaline phosphatase, liver; ALPL, Wilson disease, visceral steatosis, congenital, serpin peptidase inhibitor, clade A, member 1; SERPINA1, Huntington disease; HD, hemochromatosis, type 1; HFE1, aldehyde dehydrogenase 2 family; ALDH2, Fabry disease, major histocompatibility complex, class I, B; HLA-B, preeclampsia/eclampsia 1; PEE1, hypertension, cystic fibrosis; CF, telangiectasia, hereditary hemorrhagic, type 1; HHT1, infantile liver failure syndrome 1; ILFS1, tyrosinemia, type I; TYRSN1, Tay-Sachs disease; TSD, gap junction protein, beta-1; GJB1, apolipoprotein E; APOE, glucokinase; GCK, Menkes disease, Pendred syndrome; PDS, polycystin 1; PKD1, thyroid dyshormonogenesis 3; TDH3, HNF1 homeobox B; HNF1B, phosphorylase kinase, testis/liver, gamma-2; PHKG2, interstitial lung and liver disease; ILLD, thyroid dyshormonogenesis 2A; TDH2A, phosphorylase kinase, liver, alpha-2 subunit; PHKA2, thyroid dyshormonogenesis 1; TDH1, glucosidase, beta, acid; GBA, glutathione S-Transferase, MU-1; GSTM1, phenylketonuria; PKU, hyperphenylalaninemia, mitochondrial DNA depletion syndrome 6 (hepatocerebral type); MTDPS6, infantile sialic acid storage disease; ISSD, Sandhoff disease, C-type lectin domain family 4, member M; CLEC4M, cadherin 1; CDH1, solute carrier family 2 (facilitated glucose transporter), member 2; SLC2A2, Chanarin-Dorfman syndrome; CDS, hepatic adenomas, familial, cholestasis, progressive familial intrahepatic, 1; PFIC1, arginase 1; ARG1, peroxisome biogenesis disorder 1B; PBD1B, adrenoleukodystrophy; ALD, infantile liver failure syndrome 2; ILFS2, protein kinase C substrate, 80-KD, heavy chain; PRKCSH, Gaucher disease, GD, hepcidin antimicrobial peptide; HAMP, myoclonic epilepsy of LAFORA epilepsy, lysosomal acid lipase deficiency, Caroli disease, glycogen synthase 2; GYS2, amyloid beta A4 precursor protein; APP, hemophilia B; HEMB, liver fibrocystic disease and polydactyly, nephropathy, progressive tubulointerstitial, with cholestatic liver disease, Gallbladder disease 1; GBD1, lymphoproliferative syndrome, X-linked, 1; XLP1, phosphofructokinase, liver type; PFKL, cystic fibrosis transmembrane conductance regulator; CFTR, carnitine palmitoyltransferase I, liver; CPT1A, PKHD1 gene; PKHD1, O-phosphoserine tRNA-selenocysteine tRNA synthase; SEP SECS, cytochrome P450, family 4, subfamily F, polypeptide 2; CYP4F2, celiac disease, susceptibility to, 1; CELIAC1, Refsum disease, classic, Fanconi-Bickel syndrome; FBS, telangiectasia, hereditary hemorrhagic, type 2; HHT2, ATPase, Cu(2+)-transporting, beta polypeptide; ATP7B, interleukin 10; IL10, transforming growth factor, beta-1; TGFB1, tumor protein p53; TP53, peroxisome biogenesis disorder 1A (Zellweger); PBD1A, Alzheimer disease; AD, sphingomyelin phosphodiesterase 1, acid lysosomal; SMPD1, diabetes mellitus; DM, von Willebrand disease; VWD, chylomicron retention disease; CMRD, glucosidase, alpha, neutral AB; GANAB, Farber lipogranulomatosis; FRBRL, sickle cell anemia, hypercholesterolemia, transthyretin; TTR, Letterer-Siwe disease, hyperlipoproteinemia, graft-versus-host disease, polycystin 2; PKD2, mitochondrial complex I deficiency, inflammatory bowel disease (Crohn disease) 1; IBD1, parkin; PARK2, ATP-binding cassette, subfamily A, member 1; ABCA1, Alagille syndrome 1; ALGS1, von Hippel-Lindau syndrome; VHL, hemoglobin-beta locus; HBB, Creutzfeldt-Jakob disease; CJD, Salla disease; SD, synuclein, alpha; SNCA, cirrhosis, osteopetrosis, autosomal recessive 1; OPTB1, mucolipidosis II alpha/beta, UDP-glycosyltransferase 1 family, polypeptide A1; UGT1A1, renal cysts and diabetes syndrome; RCAD, phosphatase and tensin homolog; PTEN, immunodeficiency with hyper-IgM, type 1; HIGM1, polyglucosan body neuropathy, adult form; APBN, porphyria, congenital erythropoietic, superoxide dismutasE 1; SOD1, glycogen phosphorylase, liver; PYGL, facioscapulohumeral muscular dystrophy 1; FSHD1, insulin receptor substrate 1; IRS1, Hartnup disorder; HND, paraoxonase 1; PON1, nitric oxide synthase 3; NOS3, tumor necrosis factor; TNF, carbamoyl phosphate synthetase I; CPS1, solute carrier family 17 (acidic sugar transporter), member 5; SLC17A5, familial adenomatous polyposis 1; FAP1, Hermansky-Pudlak syndromE 1; HPS1, abetalipoproteinemia; ABL, serpin peptidase inhibitor, clade A, member 3; SERPINA3, Danon disease, Coach syndrome, glycogen phosphorylase, muscle; PYGM, NPC1 gene; NPC1, nuclear receptor subfamily 1, group H, member 3; NR1H3, hemophilia A; HEMA, peroxisome proliferator-activated receptor-gamma, coactivator 1, alpha; PPARGC1A, Tangier disease; TGD, prion protein; PRNP, peroxisome proliferator-activated receptor-gamma; PPARG, galactosidase, alpha; GLA, interleukin 6; IL6, leprosy, susceptibility to, 1; LPRS1, Friedreich ataxia 1; FRDA, glutamate pyruvate transaminase; GPT, survival of motor neuron 1; SMN1, cytochrome c oxidase, subunit 6A1; COX6A1, Charcot-Marie-Tooth disease; CMT, Parkinson disease, glutathione S-transferase, alpha-2; GSTA2, fructose-1,6-bisphosphatase 1; FBP1, adrenal hypoplasia, congenital; AHC, transmembrane protein 67; TMEM67, Darier-White disease; DAR, lecithin:cholesterol acyltransferase deficiency, growth factor, ERV1-like; GFER, gliomedin; GLDN, methionine adenosyltransferase I, alpha; MAT1A, transmembrane protein 59; TMEM59, Cowden syndrome 1; CWS1, 6-phosphofructo-2-kinase/fructose-2,6-bisphosphatase 1; PFKFB1, pleckstrin homology-like domain, family A, member 2; PHLDA2.

18. The use according to claim 15, wherein the spleen-associated disease is selected from the following list and/or relates to disfunction in the following genes: chemokine, cxc motif, receptor 4; CXCR4, Niemann-Pick disease, type C1; NPC1, Niemann-Pick disease, type D, type B, type F, type A, polycystic kidney disease 4 with or without polycystic liver disease; PKD4, hepatic fibrosis, congenital, Gaucher disease, type I, type II, perinatal, lethal, prion protein; PRNP, lysosomal acid lipase deficiency, Wolman disease, inflammatory bowel disease (Crohn disease) 1; IBD1, regional enteritis, protracted neurovisceral, glucosidase, beta, acid; GBA glucocerebrosidase pseudogene; GBAP, protein-tyrosine kinase SYK; SYK, spleen focus forming virus proviral integration oncogene SPI1; SPI1, apolipoprotein e; APOE, SICKLE CELL ANEMIA, telangiectasia, hereditary hemorrhagic, type 1; HHT1, lymphoproliferative syndrome, X-linked, 1; XLP1, Letterer-Siwe disease, cerebral arteriopathy, autosomal recessive, with subcortical infarcts and leukoencephalopathy; CARASIL, Farber lipogranulomatosis; FRBRL, hemophilia A; HEMA, erythroid-associated factor; ERAF, ceroid storage disease, nucleotide-binding oligomerization domain protein 2; NOD2, porphyria, congenital erythropoietic, histiocytosis-lymphadenopathy plus syndrome, diabetes mellitus, insulin-dependent; IDDM, osteopetrosis, autosomal recessive 1; OPTB1, glycogen storage disease Ib; GSD1B, Ia, Tangier disease; TGD, cytotoxic t lymphocyte-associated 4; CTLA4, cytotoxic t lymphocyte-associated 4, soluble isoform, right atrial isomerism; rai, polysplenia syndrome, autoimmune disease, multisystem, with facial dysmorphism; ADMFD, Kaposi sarcoma, mucopolysaccharidosis, type IVA; MPS4A, zinc finger gene in autoimmune thyroid disease 1; ZFAT1, hemoglobin-beta locus; HBB, maple syrup urine disease; MSUD, maple syrup urine disease, classic, polycystic liver disease 2 with or without kidney cysts; PCLD2, beta-glucuronidase; GUSB, neutrophilic dermatosis, acute febrile, Gillessen-Kaesb ach-Nishimura syndrome; GIKANIS, hemophagocytic lymphohistiocytosis, familial, 1; FHL1, NPC1 gene; NPC1, dehydrated hereditary stomatocytosis 1 with or without pseudohyperkalemia and/or perinatal edema; DHS1, sphingomyelin phosphodiesterase 1, acid lysosomal; SMPD1, hemochromatosis, type 1; HFE1, Gaucher disease, type III, IIIA, glycogen storage disease III; GSD3, glycogen storage disease Ma, Mb, Mc, IIId, IXa1, IXa2, VI, reticular dysgenesis, nk2 homeobox 5; NKX2-5, rh-null, regulator type; RHNR, RH-MOD, leucine zipper protein 6; LUZP6, interleukin 10; IL10, glycoprotein storage disease, granulomatous disease, chronic, X-linked; CDGX, cytochrome b-positive granulomatous disease, chronic, von Willebrand disease, type 2; VWD2, type 2A, type 2B, type 2M, type 2N, VHL gene; VHL, mast cell disease, urticaria pigmentosa, Hurler syndrome, hypermanganesemia with dystonia 1; HMNDYT1, chitinase 1; CHIT1, interleukin 6; IL6, Creutzfeldt-Jakob disease; CJD, polycystic kidney disease 1 with or without polycystic liver disease; PKD1, Alzheimer disease; AD, programmed cell death 1; PDCD1, hemochromatosis, neonatal, severe combined immunodeficiency, autosomal recessive, t cell-negative, b cell-negative, NK cell-negative, due to adenosine deaminase deficiency; SCID due to ADA deficiency, lymphoblastic leukemia, acute, with lymphomatous features; LALL, phosphatase, acid, type 5, tartrate-resistant; ACP5, serum amyloid A1; SAA1, adenosine A2A receptor; ADORA2A, leprosy, susceptibility to, 1; LPRS1, arrestin, beta, 1; ARRB1, CLN3 gene; CLN3, Sandhoff disease, Tay-Sachs disease; TSD, hexosaminidase A deficiency, adult type, included; GM2-gangliosidosis, Chanarin-Dorfman syndrome; CDS, Danon disease, Darier-White disease; DAR, Darier disease, Menkes disease, Charcot-Marie-tooth disease; CMTD, Parkinson disease PARK, melanocortin 5 receptor; MC5R, protein kinase C, delta; PRKCD, Huntington disease; HD, autoimmune lymphoproliferative syndrome, type III; ALPS3, leukemia, chronic myeloid; CML, tumor necrosis factor ligand superfamily, member 13B; TNFSF13B, prosaposin; PSAP, saposin A, lysosome-associated membrane protein 2; LAMP2, G protein-coupled receptor 183; GPR183, solute carrier family 37 (glucose-6-phosphate transporter), MEMBER 4; SLC37A4, micro rna 155; MIR155, immunity-related GTPase family, M; IRGM, chromosome 5q deletion syndrome megakaryocytes, unilobular nucleated, scavenger receptor class B, member 2; SCARB2, amyloidosis, familial visceral, transcription factor 21; TCF21, retinoic acid receptor, gamma; RARG, Omenn syndrome, Hirschsprung disease 1; HSCR1, legg-calve-pertes disease; LCPD, ceroid lipofuscinosis, neuronal, 3; CLN3, polycystic kidney disease 3, 2; PKD3, PKD2, infantile sialic acid storage disease; ISSD, adrenoleukodystrophy; ALD, adrenomyeloneuropathy, AMN, central core disease of muscle; CCD, minicore myopathy; CMT2A1, liver glycogenosis, X-linked, type II; XLG2, dementia, Lewy body; DLB, hemoglobin H disease; HBH, peroxisome biogenesis disorder 1B; PBD1B, pulmonary disease, chronic obstructive; COPD, erythrocytosis, familial, 2; ECYT2, apolipoprotein A-I; APOA1, APOA1 deficiency, myeloid zinc finger gene 1; MZF1, estrogen receptor 1; ESR1, HDL cholesterol, SH2 domain protein 2A; SH2D2A, severe combined immunodeficiency, X-linked; SCIDX1, Schimke immunoosseous dysplasia; SIOD, interferon-gamma receptor 1; IFNGR1, Glanzmann thrombasthenia; GT, galactosialidosis; GSL, solute carrier family 40 (iron-regulated transporter), member 1; SLC40A1, catenin, beta-1; CTNNB1, complement component 3; C3, C3a, C3b, C3c, C3d, acylation-stimulating protein, ASP, NK2 homeobox 3; NKX2-3, CD44 antigen; CD44, NLR family, caspase recruitment domain-containing 4; NLRC4, HFE gene; HFE, APC gene; APC, ceroid lipofuscinosis, neuronal, 10; CLN10, peroxisome proliferator-activated receptor-gamma; PPARG, caspase recruitment domain-containing protein 8; CARDS, major histocompatibility complex, class II, transactivator; MHC2TA, sphingosine-1-phosphate receptor 1; S1PR1, selenoprotein N; SELENON, interleukin 18; IL18, breakpoint cluster region; signal transducer and activator of transcription 3; STAT3, protein-tyrosine phosphatase, nonreceptor-type, 11; PTPN11, protein tyrosine phosphatase, nonreceptor-type, 22; PTPN22, vitamin D receptor; VDR, solute carrier family 29 (nucleoside transporter), member 3: SLC29A3, RAR-related orphan receptor C; RORC, chemokine, cxc motif, ligand 12; CXCL12, neuraminidase deficiency, sialidosis, type I, ring finger protein 213; RNF213, solute carrier family 11 (proton-coupled divalent metal ion transporter), member 1; SLC11A1, integrin, beta-2; ITGB2, leukocyte-associated antigens CD18/11A, CD18/11B, CD18/11C, V-KIT Hardy-Zuckerman 4 feline sarcoma viral oncogene homolog; KIT, methyl-CpG-binding protein 2; MECP2, familial mediterranean fever gene; MEFV, calpain 5; CAPN5, sorbin and SH3-domains containing protein 1; SORBS1, inhibitor of kappa light polypeptide gene enhancer in B cells, kinase of, gamma; IKBKG, caspase 3, apoptosis-related cysteine protease; CASP3, tumor necrosis factor receptor superfamily, member 11A; TNFRSF11A, placental growth factor; PGF, KIT ligand; KITLG, interleukin 18 receptor 1; IL18R1, autoimmune regulator; AIRE, chemokine, cc motif, ligand 2; CCL2, coronary artery disease.

19. The use according to claim 15, wherein the lung-associated associated disease is selected from the following list and/or relates to disfunction in the following genes: Cystic fibrosis; CF, pulmonary disease, chronic obstructive; COPD, transforming growth factor, beta-1; TGFB1, pulmonary venoocclusive disease 1, autosomal dominant; PVOD1, alpha-1-antitrypsin deficiency; A1ATAD, pulmonary venoocclusive disease 2, autosomal recessive; PVOD2, surfactant metabolism dysfunction, pulmonary, 2; SMDP2, pulmonary hypertension, primary; PPH, lung cancer, alveolar cell carcinoma, including: adenocarcinoma of lung, non-small cell lung cancer, lung cancer, surfactant metabolism dysfunction, pulmonary, 1; SMDP1, surfactant, pulmonary-associated protein c; SFPC, surfactant metabolism dysfunction, pulmonary, 3; SMDP3, bone morphogenetic protein receptor, type II; BMPR2, Mowat-Wilson Syndrome; MOWS, Noonan syndrome 1; NS1, Pterygium Colli syndrome, pulmonary fibrosis, idiopathic; IPF, Hamman-Rich disease, Niemann-Pick Disease, surfactant, pulmonary-associated protein A1; SFTPA1, interferon-related developmental regulator 1; IFRD1, telangiectasia, hereditary hemorrhagic, type 1; HHT1, serpin peptidase inhibitor, CLADE A, MEMBER 1; SERPINA1, dyskeratosis congenita, autosomal dominant 1; DKCA1, surfactant, pulmonary-associated protein B; SFTPB, microphthalmia, syndromic 9; MCOPS9, lymphoma, Hodgkin, classic; CHL, pulmonary fibrosis and/or bone marrow failure, telomere-related, 1; PFBMFT1, pulmonary hypertension, primary, 4; pph4, hedgehog-interacting protein; HHIP, surfactant metabolism dysfunction, pulmonary, 5; SMDP5, cystic disease of lung, asthma, total anomalous pulmonary venous return 1; TAPVR1, sodium channel, nonvoltage-gated 1, alpha subunit; SCNN1A, tetralogy of fallot; TOF, sickle cell anemia, NK2 homeobox 1; NKX2-1, Parkinson disease; PD, fibroblast growth factor 10; FGF10, Keutel syndrome; KTLS, IgE responsiveness, atopic; IGER, pulmonic stenosis, aplasia of lacrimal and salivary glands; ALSG, cirrhosis, jagged 1; JAG1, interstitial pneumonitis, desquamative, familial; DIP, telangiectasia, hereditary hemorrhagic, type 2; HHT2, pulmonary arterial hypertension, granulomatous disease, chronic, X-linked; CDGX, tumor protein p53; TP53, Gaucher disease, type I, elastin; ELN, sphingomyelin phosphodiesterase 1, acid lysosomal; SMPD1, Fabry disease, pulmonic stenosis and deafness, major histocompatibility complex, class I, B; HLA-B, neurofibromatosis, type I; NF1, rheumatoid arthritis; RA, Noonan syndrome 8; NS8, Hermansky-Pudlak syndrome 1; HPS1, TSC1 gene; TSC1, Young syndrome, V-KI-RAS2 Kirsten RAT sarcoma viral oncogene homolog; KRAS, velocardiofacial syndrome, surfactant metabolism dysfunction, pulmonary, 4; SMDP4, Williams-Beuren syndrome; WBS, mesothelioma, malignant; MESOM, epidermal growth factor receptor; EGFR, surfactant, pulmonary-associated protein D; SFTPD, Marfan syndrome; MFS, glucosidase, beta, acid; GBA, protein-tyrosine phosphatase, nonreceptor-type, 11; PTPN11, Alagille syndrome 1; ALGS1, atrial septal defect 1; ASD1, thyroid dyshormonogenesis 2A; TDH2A, Costello syndrome; CSTLO, major histocompatibility complex, class II, DR beta-1; HLA-DRB1, Smith-Lemli-Opitz syndrome; SLOS, arthrogryposis, distal, type 5; DA5, lymphangioleiomyomatosis; LAM, glycogen storage disease, GSD, supravalvular aortic stenosis; SVAS, heart defects, congenital, and other congenital anomalies; HDCA, telomerase ma component; TERC, right atrial isomerism; RAI, interstitial lung and liver disease; ILLD, activin A receptor, type II-like 1; ACVRL1, facioscapulohumeral muscular dystrophy 1; FSHD1, major histocompatibility complex, class II, DP beta-1; HLA-DPB1, Hermansky-Pudlak Syndrome 5; HPS5, breast cancer, central hypoventilation syndrome, congenital; CCHS, neurofibromin 1; NF1, severe combined immunodeficiency, lysosomal acid lipase deficiency, endothelin receptor, type B; EDNRB, matrix metalloproteinase 1; MMP1, tuberous sclerosis 1; TSC1, signal transducer and activator of transcription 3; STAT3, caveolin 1; CAV1, lung cancer susceptibility 1; LNCR1, Li-Fraumeni syndrome; LFS, pleuropulmonary blastoma; PPB, prostaglandin-endoperoxide synthase 2; PTGS2, hypertrophic osteoarthropathy, primary, autosomal recessive, 1; PHOAR1, cutis laxa, autosomal dominant 1; ADCL1, alpha-2-macroglobulin; A2M, dyskeratosis congenita, autosomal dominant 3; DKCA3, bronchiectasis with or without elevated sweat chloride 1; BESC1, patent ductus arteriosus 1; PDA1, muscleblind-like splicing regulator 1; MBNL1, telomerase reverse transcriptase; TERT, heterotaxy, visceral, 1, X-linked; HTX1, Birt-Hogg-Dube syndrome; BHD, lectin, mannose-binding, soluble, 2; MBL2, nephrotic syndrome, type 1; NPHS1, spinal muscular atrophy, type I; SMA1, phosphatase and tensin homolog; PTEN, Leopard syndrome 1; LPRD1, matrix metalloproteinase 9; MMP9, choreoathetosis and congenital hypothyroidism with or without pulmonary dysfunction; CAHTP, dyskeratosis congenita, X-linked; DKCX, Hoyeraal-Hreidarsson syndrome, toll-like receptor 4; TLR4, cutis laxa, autosomal recessive, type IC; ARCL1C, pulmonary function, immunoglobulin a deficiency 1; IGAD, lung agenesis, nitric oxide synthase 3; NOS3, interleukin 10; IL10, Letterer-Siwe disease, tuberous sclerosis 2; TSC2, chemokine, cc motif, ligand 2; CCL2, solute carrier family 6 (neurotransmitter transporter, serotonin), member 4; SLC6A4, lung cancer susceptibility 4; LNCR4, serpin peptidase inhibitor, clade A, member 3; SERPINA3, mucin 5, subtype B, tracheobronchial; MUCSB, amyotrophic lateral sclerosis; ALS, interleukin 13; IL13, dermatitis, atopic; ATOD, bpi fold-containing protein, family a, member 1; BPIFA1, Farber lipogranulomatosis; FRBRL, vascular endothelial growth factor A; VEGFA, Holt-Oram syndrome; HOS, granulomatous disease, chronic, autosomal recessive, cytochrome b-positive, type II; CDG2, minicore myopathy with external ophthalmoplegia, diaphragmatic hernia, DIH, cutis laxa, autosomal recessive, type IA; ARCL1A, chemokine, CC motif, receptor 5; CCR5, fibrillin 1; FBN1, FAS ligand; FASLG, interleukin 17A; IL17A, complement hyperactivation, angiopathic thrombosis, and protein-losing enteropathy; CHAPLE, brain small vessel disease with or without ocular anomalies; BSVD, presenilin 2; PSEN2, collagen, type i, alpha-1; COL1A1, bleomycin hydrolase; BLMH, forkhead box O3A; FOXO3A), ciliary dyskinesia, primary, 33; CILD33 (secretoglobin, family 1A, member 1; SCGB1A1) ataxia-telangiectasia; AT, pulmonary alveolar proteinosis, acquired, Loeys-Dietz syndrome 2; LDS2, angiotensin I-converting enzyme 2; ACE2, delta-like 4; DLL4, mitral valve prolapse 1; MVP1, Waardenburg syndrome, type 4A; WS4A, pulmonary hypertension, primary, 2; PPH2, ciliary dyskinesia, primary, 1; CILD1, rigid spine muscular dystrophy 1; RSMD1, pulmonary fibrosis and/or bone marrow failure, telomere-related, 4; PFBMFT4, Von Hippel-Lindau syndrome; VHL, pulmonary fibrosis and/or bone marrow failure, telomere-related, 3; PFBMFT3.

20. The use according to claim 15, wherein the heart-related disease manifest itself in high blood pressure, heart attacks, heart failure, and stroke and transient ischemic attack (TIA) and is associated with disfunction of one or combination of the following genes: IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCGS (ATP-binding cassette, sub-family G (WHITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), VWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member 11b), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1 A (hypoxia inducible factor 1, alpha subunit (basic helix-loop-helix transcription factor)), CXCR4 (chemokine (C-X-C motif) receptor 4), PROC (protein C (inactivator of coagulation factors Va and VIIIa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomerase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CABIN1 (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IL17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, A1 polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member 1B), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac1)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin L1), PCNA (proliferating cell nuclear antigen), IGF2 (insulin-like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALCA (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PECAM1 (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YME1-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-.beta.-methyltransferase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (B321 placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHC1 (SHC (Src homology 2 domain containing) transforming protein 1), LRP1 (low density lipoprotein receptor-related protein 1), SOCS3 (suppressor of cytokine signaling 3), ADH1B (alcohol dehydrogenase 1 B (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor H1), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQO1 (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCHE (butyrylcholinesterase), LIPE (lipase, hormone-sensitive), PRDXS (peroxiredoxin 5), ADORA1 (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CHGA (chromogranin A (parathyroid secretory protein 1)), IAPP (islet amyloid polypeptide), RHO (rhodopsin), ENPP1 (ectonucleotide pyrophosphatase/phosphodiesterase 1), PTHLH (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISH (cytokine inducible SH2-containing protein), GAST (gastrin), MYOC (myocilin, trabecular meshwork inducible glucocorticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), HSF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTH (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOH (apolipoprotein H (beta-2-glycoprotein I)), S100A8 (S100 calcium binding protein A8), IL11 (interleukin 11), ALOX15 (arachidonate 15-lipoxygenase), FBLN1 (fibulin 1), NR1H3 (nuclear receptor subfamily 1, group H, member 3), SCD (stearoyl-CoA desaturase (delta-9-desaturase)), GIP (gastric inhibitory polypeptide), CHGB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), HSD11B2 (hydroxysteroid (11-beta) dehydrogenase 2), CALCRL (calcitonin receptor-like), GALNT2 (UDP-N-acetyl-alpha-D-galactosamine:polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adenovirus E1B 19 kDa interacting protein 3), GCKR (glucokinase (hexokinase 4) regulator), S100A12 (S100 calcium binding protein A12), PADI4 (peptidyl arginine deiminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), IDDM2 (insulin-dependent diabetes mellitus 2), RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-1(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (F11 receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), KHK (ketohexokinase (fructokinase)), SAT1 (spermidine/spermine N1-acetyltransferase 1), GGH (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RHOB (ras homolog gene family, member B), LY96 (lymphocyte antigen 96), FOXO1 (forkhead box 01), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPR1 (nuclear protein, transcriptional regulator, 1), 15-Sep(15 kDa selenoprotein), CILP2 (cartilage intermediate layer protein 2), TERC (telomerase RNA component), GGT2 (gamma-glutamyltransferase 2), MT-CO1 (mitochondrially encoded cytochrome c oxidase I), and UOX (urate oxidase, pseudogene), Pon1 (paraoxonase 1), LDLR (LDL receptor), ApoE (Apolipoprotein E), Apo B-100 (Apolipoprotein B-100), ApoA (Apolipoprotein(a)), ApoA1 (Apolipoprotein A1), CBS (Cystathione B-synthase), Glycoprotein IIb/IIb, MTHRF (5,10-methylenetetrahydrofolate reductase (NADPH), Cacna1C, Sod1, Pten, Ppar(alpha), Apo E, Leptin, and combinations thereof.

21. The use according to claim 15, wherein the kidney-associated disease is selected from: Bartter's syndrome, Gitelman syndrome, nephrolithiasis. renal amyloidosis, hypertension; primary aldosteronism; Addison's disease; renal failure; glomerulonephritis; chronic glomerulonephritis: tubulointerstitial nephritis; cystic disorders of the kidney and dysplastic malformations such as polycystic disease, renal dysplasias, and cortical or medullary cysts; inherited polycystic renal diseases (PRD), such as recessive and autosomal dominant PRD; medullary cystic disease; medullary sponge kidney and tubular dysplasia; Alport's syndrome; non-renal cancers which affect renal physiology, such as bronchogenic tumors of the lungs or tumors of the basal region of the brain; multiple myeloma; adenocarcinomas of the kidney; metastatic renal carcinoma; in addition, nephrotoxic disorders include any functional or morphologic change in the kidney produced by any pharmaceutical, chemical, or biological agent that is ingested, injected, inhaled, or absorbed. Affected genes belong to the group consisting of uromodulin, NKCC2 (bumetanide-sensitive Na—K—Cl cotransporter 2), NCCT (thiazine-sensitive Na—Cl cotransporter), aldolase B, ROMK1 (inwardly-rectifying voltage-gated K channel), ATP1G1 (Na—K ATPase gamma subunit), PDZK1 (PDZ domain-containing protein), NPT-1 (Na-dependent phosphate cotransporter), calbindin, kininogen, and CIC-Kb (chloride channel).
